(19) United States
(12) Patent Application Publication
(10) Pub. No.: US 2015/0127104 A1
(43) Pub. Date: May 7, 2015

(54) IN-SITU FORMATION OF A JOINT REPLACEMENT PROSTHESIS

(71) Applicant: ULTIMATE JOINT LTD., Oshrat (IL)

(72) Inventors: Ofer Levy, Ramat Meir (IL); Yael Sonnino-Levy, Ramot Meir (IL); Mark Shahar, Holon (IL); Nir Barkai, Kfar Sava (IL)

(73) Assignee: ULTIMATE JOINT LTD. a corporation

(21) Appl. No.: 14/397,515

(22) PCT Filed: May 2, 2013
(86) PCT No.: PCT/IL2013/050376

§ 371 Date: Oct. 28, 2014

Related U.S. Application Data
(60) Provisional application No. 61/641,901, filed on May 3, 2012.

(51) Int. Cl. A61F 2/30; A61F 2/46; A61F 2/40; A61B 17/16

(52) U.S. Cl. CPC A61F 2/30756; A61B 17/1684; A61F 2/4612; A61F 2/40; A61F 2002/4635; A61F 2002/30583; A61F 2002/4619; A61F 2002/30125; A61F 2002/30242
USPC 623/14.12

(57) ABSTRACT

An expandable mold configured to be arthroscopically introduced into a joint for in-situ formation of a joint replacement prosthesis, and a flowable, curable substance configured for forming the joint replacement prosthesis inside said mold. In addition, a surgical kit for arthroscopic, in-situ formation of a joint replacement prosthesis, the surgical kit comprising: an expandable prosthesis mold configured to be arthroscopically introduced into a joint; at least one arthroscopic instrument configured to form an ellipsoidal cavity between two interfacing bones of the joint, for receiving said mold; and a first flowable, curable substance configured for forming the prosthesis inside said mold.

### FIELD OF THE INVENTION

The invention relates to in-situ formation of a joint replacement prosthesis.

### BACKGROUND OF THE INVENTION

Joint surfaces are subject to damage by injury and inflammation, but most commonly by arthritis. Arthritis affects the articular cartilage, sometimes to a degree which exposes and roughens the underlying bone surfaces, thereby creating mechanical interference to the smooth motion of the joint, severe pain and general joint dysfunction.

One of today's most successful approaches for treating damaged articular cartilage is total joint replacement, also referred to as total joint arthroplasty. The deformed joint surfaces are resected and replaced by one or more artificial prostheses, re-enabling smooth and normal joint motion. In existence today are various techniques for performing total joint replacement or related arthoplastic treatments. Some examples are discussed below.

U.S. Pat. No. 6,248,131 to Felt et al. discloses a method and related materials and apparatus for using minimally invasive means to repair (e.g., reconstruct) tissue such as fibrocartilage, and particularly fibrocartilage associated with diarthroidal and amphiarthroidal joints. The method involves the use of minimally invasive means to access and prepare damaged or diseased fibrocartilage within the body, and to then deliver a curable biomaterial, such as a two-part polyurethane system, to the prepared site, and to cure the biomaterial in situ in order to repair the fibrocartilage. Applications include repair and replacement of the intervertebral disc of the spine.

U.S. Pat. No. 6,443,988 to Felt et al. discloses a method, and related composition and apparatus for repairing a tissue site. The method involves the use of a curable polyurethane biomaterial composition having a plurality of parts adapted to be mixed at the time of use in order to provide a flowable composition and to initiate cure. The flowable composition can be delivered using minimally invasive means to a tissue site and once delivered fully cured to provide a permanent and biocompatible prosthesis for repair of the tissue site. Further provided are a mold apparatus, e.g., in the form of a balloon or tubular cavity, for receiving a biomaterial composition, and a method for delivering and filling the mold apparatus with a curable composition in situ to provide a prosthesis for tissue repair.

U.S. Pat. No. 7,758,649 to Walsh et al. discloses an implant for positioning within a particularly dimensioned body cavity. The implant is reversibly deformable between an expanded state and a compressed state. The implant is constructed and arranged for insertion within the body cavity when in its compressed state, and pressurelessly conforms to the cavity dimensions in its expanded state. Particularly, the implant is characterized by spontaneous deformation to the expanded state in situ within the body cavity while retaining and/or absorbing at least one flowable constituent as a function of its degree of deformation.

U.S. Patent Application Publication No. 2005/0043808 Felt et al. discloses a method, and related composition and apparatus for repairing a tissue site. The method involves the use of a curable polyurethane biomaterial composition having a plurality of parts adapted to be mixed at the time of use in order to provide a flowable composition and to initiate cure. The flowable composition can be delivered using minimally invasive means to a tissue site and upon delivery fully cured providing a permanent and biocompatible prosthesis for repair of the tissue site. Further provided are a mold apparatus, e.g., in the form of a balloon or tubular cavity, for receiving a biomaterial composition, and a method for delivering and filling the mold apparatus with a curable composition in situ to provide a prosthesis for tissue repair.

U.S. Patent Application Publication No. 2005/0229433 to Cachia discloses methods and devices for manipulating alignment of the foot to treat patients with flat feet, posterior tibial tendon dysfunction and metatarsophalangeal joint dysfunction. An inflatable implant is positioned in or about the sinus tarsi and/or first metatarsal-phalangeal joint of the foot. The implant is insertable by minimally invasive means and inflatable through a catheter or needle. Inflation of the implant alters the range of motion in the subtalar or first metatarsal-phalangeal joint and changes the alignment of the foot.

U.S. Patent Application Publication No. 2009/0287309 to Walch et al. discloses a method for implanting an intra-articular shoulder prosthesis. The method includes removing a proximal portion of a humerus. The proximal portion of the humerus preferably forms a resected portion. The resected portion has a convex outer surface and an inner surface. The method further includes engaging the convex outer surface of the resected portion with a cut surface of the proximal portion of the humerus. The cut surface of the proximal portion of the humerus and/or the inner surface of the resected portion are optionally processed to form a generally concave surface, such as by impacting. In one embodiment, the inner surface of the resected portion is impacted into engagement with the cut surface of the proximal portion of the humerus. The generally concave inner surface of the resected portion forms a concave articular surface to receive an interpositional implant.

U.S. Patent Application No. 2010/0241152 to Tilson et al. discloses inflatable medical devices and methods for making and using the same. The inflatable medical devices can be medical balloons. The balloons can be configured to have a through-lumen or no through-lumen and a wide variety of geometries. The device can have a high-strength, non-compliant, fiber-reinforced, multi-layered wall. The inflatable medical device can be used for angioplasty, kyphoplasty, percutaneous aortic valve replacement, or other procedures described herein.

U.S. Pat. No. 8,100,979 to Felt. et al. method and system for the creation or modification of the wear surface of orthopedic joints, involving the preparation and use of one or more partially or fully preformed and procured components, adapted for insertion and placement into the body and at the joint site. In a preferred embodiment, component(s) can be partially cured and generally formed ex vivo and further and further formed in vivo at the joint site to enhance conformance and improve long term performance. In another embodiment, a preformed balloon or composite material can be inserted into the joint site and filled with a flowable biomaterial in situ to conform to the joint site. In yet another embodiment, the preformed component(s) can be fully cured and formed ex vivo and optionally further fitted and secured at the joint site. Preformed components can be sufficiently pliant to permit insertion through a minimally invasive portal, yet resilient enough to substantially assume, or tend towards, the desired form in vivo with additional forming there as needed.

Finally, PCT Publication No. WO2010/107949 to Nikolchev et al. discloses a method for creating space in a joint, the method comprising: applying force to a body part so as to distract the joint and create an intrajoint space; inserting an expandable member into the intrajoint space while the expandable member is in a contracted condition; expanding the expandable member within the intrajoint space; and reducing the force applied to the body part so that the joint is supported on the expandable member.

A significant number of orthopedic surgeries are performed today using minimally-invasive techniques, such as arthroscopy. In arthroscopic surgery, examination and sometimes treatment of the joint are performed by inserting an arthroscope through a small incision. Additional surgical instruments may be inserted into the joint via other small incisions. Arthroscopy often reduces recovery time and minimizes trauma to connective tissue as well as external scarring.

There is still a need in the art for enhanced surgical methods, devices and kits for minimally-invasive joint replacement.

### SUMMARY OF THE INVENTION

There is provided, in accordance with some embodiments, a surgical kit for arthroscopic, in-situ formation of a joint replacement prosthesis, the surgical kit comprising: an expandable prosthesis mold configured to be arthroscopically introduced into a joint; at least one arthroscopic instrument configured to form an ellipsoidal cavity between two interfacing bones of the joint, for receiving said mold; and a first flowable, curable substance configured for forming the prosthesis inside said mold.

In some embodiments, said mold is characterized by a smooth, ellipsoidal inner surface, such that an outer surface of said prosthesis, when formed, is smooth and ellipsoidal.

In some embodiments, said ellipsoidal inner surface comprises a spheroidal inner surface.

In some embodiments, said spheroidal inner surface comprises a spherical inner surface.

In some embodiments, said mold is characterized by a final inflated size.

In some embodiments, said mold comprises a balloon made of a rigid material.

In some embodiments, said mold is made of a non-elastic material.

In some embodiments, the surgical kit further comprises an expandable spacer configured to be arthroscopically introduced into the joint, wherein said spacer is configured, when expanded, to maintain the ellipsoidal cavity between the two interfacing bones at least during the formation of the prosthesis.

In some embodiments, said mold is provided within said spacer, such that said mold and said spacer are configured to be arthroscopically introduced into the joint together.

In some embodiments, said spacer is characterized by a final inflated size.

In some embodiments, said mold and said spacer, when at their final inflated size, are isomorphic.

In some embodiments, said first flowable, curable substance is further configured for forming a prosthetic layer over the prosthesis, inside said spacer.

In some embodiments, the surgical kit further comprises a second flowable, curable substance configured for forming a prosthetic layer over the prosthesis, inside said spacer.

In some embodiments, said spacer is characterized by a smooth, ellipsoidal inner surface, such that an outer surface of said prosthetic layer, when formed, is smooth and ellipsoidal.

In some embodiments, the surgical kit further comprises a pumping system configured to control inflation of said mold.

In some embodiments, the surgical kit further comprises a pumping system configured to control injection of said substance into said mold.

In some embodiments, the surgical kit further comprises a pumping system configured to control inflation of said spacer.

In some embodiments, the surgical kit further comprises a pumping system configured to control injection of at least one of said substance and said different substance into said spacer.

In some embodiments, the surgical kit further comprises an arthroscopic extraction instrument configured to extract said mold after the prosthesis is formed.

In some embodiments, said arthroscopic extraction instrument is further configured to extract said spacer after at least one of the prosthesis and the prosthetic layer is formed.

In some embodiments, the arthroscopic extraction instrument comprises at least one wire, for example, a rigid thin wire. In some embodiments, at least part of the wire is attached to a surface of the mold or embedded in the mold. In some embodiments, at least part of the wire is attached to a surface of the spacer or embedded in the spacer. In some embodiments, the wire is configured to rip at least part of the mold along the path of the wire upon pulling said wire. In some embodiments, the wire is configured to rip at least part of the spacer along the path of the wire upon pulling said wire.

In some embodiments, the surgical kit further comprises a guide wire configured to guide surgical tools into said joint.

In some embodiments, the surgical kit further comprises starter drill configured to drill an initial hole in the joint.

In some embodiments, said starter drill comprises an adjustable stopper configured to allow drilling up to a preset depth.

In some embodiments, said starter drill comprises a convex end surface.

In some embodiments, the surgical kit further comprises a convex, expandable reamer configured to form the ellipsoidal cavity between the two bones.

In some embodiments, the surgical kit further comprises a guide cannula configured to be secured relative to the joint to guide said guide wire into the joint at a predetermined angle.

In some embodiments, said guide wire is further configured to guide said reamer into the joint at the predetermined angle over said guide wire.

In some embodiments, said reamer is configured, when forming the ellipsoidal cavity, to ream one of the two bones.

In some embodiments, said reamer is configured, when forming the ellipsoidal cavity, to ream the two bones.

In some embodiments, said arthroscopic guide cannula comprises concave, collapsible arms for positioning said guide wire relative to the joint.

In some embodiments, the joint is a ball-and-socket joint and said arms are configured to cling to the ball of the ball-and-socket joint.

In some embodiments, said starter drill is a cannulated starter drill.

In some embodiments, said mold comprises a fluid port configured to extend externally to the joint when said mold is introduced into the joint.

In some embodiments, said spacer comprises a fluid port configured to extend externally to the joint when said spacer is introduced into the joint.

In some embodiments, the surgical kit further comprises a file configured to remove cured substance protruding from said mold due to curing of the substance in said fluid port of said mold.

In some embodiments, said file is further configured to remove cured substance protruding from said spacer due to curing of the substance in said fluid port of said spacer.

In some embodiments, the surgical kit further comprises an ultraviolet (UV) curer for curing said substance inside said joint.

There is further provided, in accordance with some embodiments, a minimally-invasive method for in-situ formation of a joint replacement prosthesis, the method comprising: arthroscopically forming an ellipsoidal cavity between two interfacing bones of the joint; and arthroscopically forming an ellipsoidal joint replacement prosthesis in the cavity.

In some embodiments, the forming of said ellipsoidal joint replacement prosthesis comprises: arthroscopically introducing an expandable prosthesis mold into said cavity; and injecting a first flowable, curable substance into said mold, thereby forming said ellipsoidal joint replacement prosthesis inside said mold.

In some embodiments, the forming of the ellipsoidal cavity comprises reaming at least one of the two interfacing bones using a convex, expandable reamer.

In some embodiments, the method further comprises drilling an initial hole in the joint, to allow introduction of said convex, expandable reamer into the joint.

In some embodiments, the method further comprises, prior to injecting said substance: arthroscopically introducing an expandable spacer into the joint; and expanding said spacer to maintain the ellipsoidal cavity between the two interfacing bones at least during the formation of the prosthesis.

In some embodiments, said mold is provided within said spacer, such that said mold and said spacer are introduced together, arthroscopically, into the cavity.

In some embodiments, the method further comprises forming injecting the first flowable, curable substance into said spacer, to form a prosthetic layer over said prosthesis.

In some embodiments, the method further comprises injecting a second flowable, curable substance into said spacer, to form a prosthetic layer over said prosthesis.

In some embodiments, the method further comprises extracting said mold after said prosthesis is formed.

In some embodiments, the method further comprises extracting said spacer after said prosthetic layer is formed.

In some embodiments, the method further comprises applying UV radiation for curing said first substance.

In some embodiments, the method further comprises applying UV radiation for curing said second substance.

In some embodiments, the method further comprises removing cured substance protruding from said mold.

In some embodiments, the method further comprises prior to arthroscopically forming an ellipsoidal cavity, arthroscopically releasing the joint capsule of the joint.

There is further provided, in accordance with some embodiments, a corrective surgical method comprising modifying a bone being the ball of a ball-and-socket joint to have a socket shape, and forming, in-situ, an ellipsoidal prosthesis between said bone and a different bone being the socket of said ball-and-socket joint.

There is further provided, in accordance with some embodiments, an expandable mold configured to be arthroscopically introduced into a joint for in-situ formation of a joint replacement prosthesis, and a flowable, curable substance configured for forming the joint replacement prosthesis inside said mold.

There is further provided, in accordance with some embodiments, a joint replacement prosthesis assembly for in-situ formation of a joint replacement prosthesis, the assembly comprising a plurality of parts, each being sized so as to enable its minimally-invasive introduction into a damaged joint, wherein said plurality of parts are configured to be assembled into the joint replacement prosthesis.

In some embodiments, said plurality of parts comprises a core part and multiple peripheral parts configured to be mounted onto said core part.

In some embodiments, said plurality of parts comprises a plurality of similarly-shaped parts.

In some embodiments, the joint replacement prosthesis assembly further comprises one or more securing elements configured to secure said plurality of parts once assembled.

In some embodiments, said one or more securing elements comprises one or more bolts. The present invention further provides, according to some embodiments, an expandable prosthesis mold configured to be arthroscopically introduced into a joint; wherein the mold is configured to allow in-situ formation of a joint replacement prosthesis within the mold; wherein the mold is configured to have a collapsed form and an expanded form; wherein the expanded form has a final size; wherein the mold comprises at least one wire, wherein at least a part of the wire is attached to a surface of the mold or embedded in the mold, the wire having a proximal end and a distal end; and wherein at least part of the mold is configured to rip along the path of said wire upon pulling the proximal end of said wire.

In some embodiments, the mold is an inflatable mold. In some embodiments, the mold further comprises a string configured to extract the mold from a subject's body upon pulling said string.

In some embodiments, the proximal end of said wire is configured to extend into an arthroscopic extraction instrument. In some embodiments, the wire is made of a material configured to transmit energy, such as, but not limited to an electricity conducting material. In some embodiments, the wire is configured to connect into an energy source, such as, but not limited to, an electricity source.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1 shows a healthy human shoulder joint;

FIG. 2 shows a damaged human shoulder joint;

FIGS. 3A-B shows the damaged joint and a guide cannula;

FIG. 3Ba shows the damaged joint and a guide wire;

FIG. 3C shows the guide wire and a starter drill used in drilling an initial bore in the damaged joint;

FIG. 3D shows the guide wire and a convex, expandable reamer used in resecting articular matter for forming a cavity in the damaged joint;

FIG. 3E shows a spheroidal cavity formed between the interfacing bones and within the bones themselves;

FIG. 3F shows an expandable joint replacement prosthesis mold;

FIG. 4 shows a flow chart of a method for in-situ formation of a joint replacement prosthesis;

FIGS. 5A-C show a guide cannula in cross-sectional and perspective views with collapsed (A) and expanded (B, C) securing elements;

FIGS. 6A-F show a cannulated starter drill in perspective, side and cross-sectional views;

FIGS. 7A-D show a convex, cannulated expandable reamer in its collapsed form in front, cross-sectional, side and isometric views;

FIGS. 7E-H show the convex, cannulated expandable reamer in its expanded form in front, cross-sectional, side and isometric views;

FIGS. 8A-C show cross-sectional views of a joint replacement prosthesis mold and an insertion instrument;

FIGS. 9A-B show cross-sectional views of the mold and an optional spacer containing it;

FIG. 10 shows a perspective view of three molds of gradual sizes provided one inside the other;

FIG. 11 shows a first exemplary extraction instrument;

FIG. 12 shows a second exemplary extraction instrument;

FIGS. 13A-D show a third exemplary extraction instrument;

FIGS. 14A-E show a fourth exemplary extraction instrument;

FIGS. 15A-E show a fifth exemplary extraction instrument; and

FIG. 16 shows another option for a joint replacement prosthesis.

### DETAILED DESCRIPTION

A surgical method for arthroscopic, in-situ formation of a joint replacement prosthesis, as well as a surgical kit for facilitating the same, are disclosed herein. Advantageously, the method may be performed entirely in a minimally-invasive manner, such as using arthroscopic techniques and instrumentation. An expandable, optionally inflatable, prosthesis mold may be arthroscopically introduced into a damaged joint, and a smooth, ellipsoidal joint replacement prosthesis may then be formed in-situ, by filling the mold with a suitable flowable, curable substance. The ellipsoidal mold may be, more specifically, of a spheroidal shape, or even more specifically, a spheroid—according to the medical needs. Optionally, the prosthesis mold may be an ellipsoid, where the difference between the apogee and perigee of the ellipsoid is, for example, about 1 millimeter. Optionally, the prosthesis mold may be spherical or a spheroid. Each possibility represents a separate embodiment of the present invention. Optionally, the mold is made of a non-elastic material. The mold may be rigid or partially rigid (for example, may include rigid parts or may include rigid parts that may form a rigid mold when assembles). The mold may be made of a material with zero or partial compliance.

The damaged joint may be adapted and/or modified for receiving the prosthesis mold by resecting cartilage and/or bone material from one or two of the interfacing bones of the joint using arthroscopic instrumentation, to form an ellipsoidal, smooth cavity for the prosthesis. In a ball-and-socket type joint, such as the shoulder or the hip joints, the prosthesis mold becomes the ball part of the joint, while the humeral or femoral head, respectively, becomes a socket. The ellipsoidal joint replacement prosthesis then serves as a ball interfacing between the original glenoid or acetabulum socket and the newly-created humeral or femoral head socket, respectively, allowing them to slide smoothly on its outer surface. According to some embodiments, the prosthesis slides only or mainly against the humerus. Each possibility represents a separate embodiment of the present invention. According to other embodiments, the prosthesis slides only or mainly against the glenoid. Each possibility represents a separate embodiment of the present invention. According to other embodiments, the prosthesis slides against both the humerus and the glenoid. According to some embodiments, the prosthesis slides against the humerus and/or the glenoid so as long as normal and/or maximal movement range of the joint is maintained. Each possibility represents a separate embodiment of the present invention.

It is to be noted, that according to various embodiments of the present invention, the cavity and/or joint replacement prosthesis and/or mold may be of an ellipsoid, spheroid or spherical shape. Each possibility represents a separate embodiment of the present invention.

According to some embodiments, the cavity and/or joint replacement prosthesis and/or mold may be spherical.

Following the arthroscopic, in-situ formation of the joint replacement prosthesis, the prosthesis remains enclosed within the joint capsule and the rest of the soft tissues, namely, tendons, ligaments and/or the muscles around the joint. The tension of the soft tissues may keep the new joint replacement prosthesis in place. However, the area that is reamed from the bone may become covered with new fibrocartilage which articulates with the new ball joint.

Advantageously, as discussed above, the method is performed arthroscopically, optionally without cutting any tendons or muscles in order to get into the joint. Therefore, applying the method eliminates the need to protect or repair tendons and muscles following the procedure. Thus, recovery from the present joint replacement procedure is extremely short. In fact, immediately following the procedure, a patient can start with a mobilization and rehabilitation exercise program. Thus, the procedure provides accelerated rehabilitation and recovery, which are much shorter than rehabilitation and recovery after standard joint replacement procedures.

The aforementioned advantages render the method suitable for ambulatory surgery centers (also known as “outpatient surgery centers” or “same-day surgery centers”).

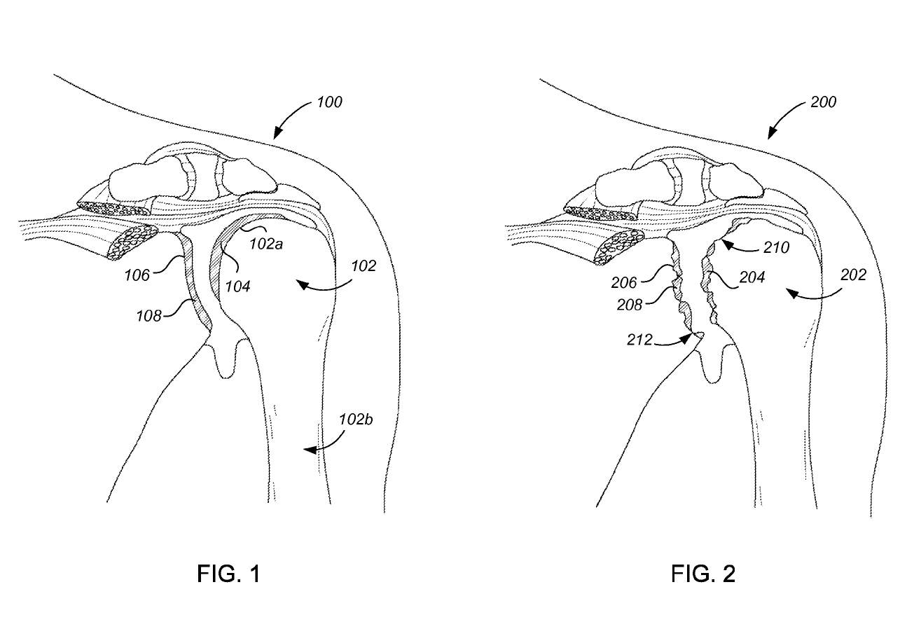

Reference is now made to FIG. 1, which shows a schematic illustration of a healthy human shoulder joint 100. The shoulder joint is used as a demonstrative joint throughout the disclosure only for reasons of simplicity; the various discussions made with reference to this demonstrative joint apply, mutatis mutandis, to other mammalian joints, such as the hip, knee, wrist, ankle, metacarpophalangeal, metatarsophalangeal, interphalangial joint and others. Shoulder joint 100 exhibits smooth, healthy joint surfaces: a humerus head 102 and its cartilage cover 104, as well as a glenoid cavity 106 and its cartilage cover 108, are all shown essentially intact.

FIG. 2 shows a schematic illustration of a representative damaged joint, a human shoulder joint 200. The damage to the various parts of damaged joint 200 is shown by way of example; in practice, joint damage may be exhibited in various patterns of articular surface deformation. The term “articular surface” (or “joint surface”), as referred to herein, may relate to the external surface of the cartilage cover of each of the bones of a joint. The external surface of the bone, which is either exposed or still covered by cartilage, may also be referred to as an articular surface; its relevancy arises in cases where cartilage damage causes a bone to be partially exposed.

A cartilage cover 204 of a humerus head 202 is shown damaged, and so is a cartilage cover 208 of a glenoid cavity 206. Pain may be caused as cartilage covers 204 and 208 contact each other during joint motion. Worse, exposed areas 210 of humerus head 202 and/or exposed areas 212 of glenoid cavity 206 may also come in contact with one another and/or with an opposing cartilage cover, causing further damage.

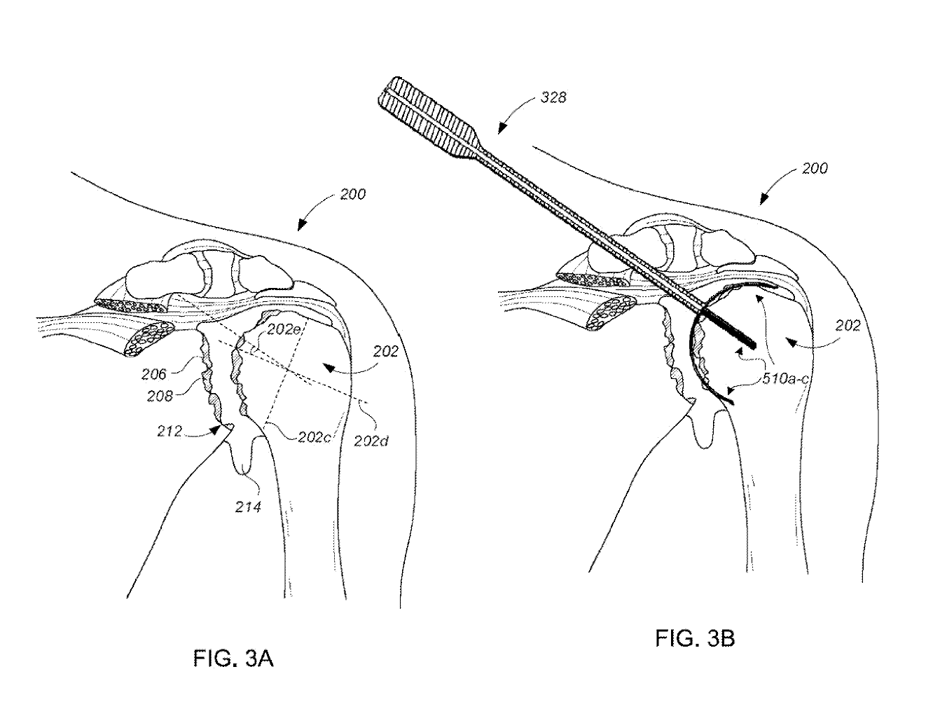

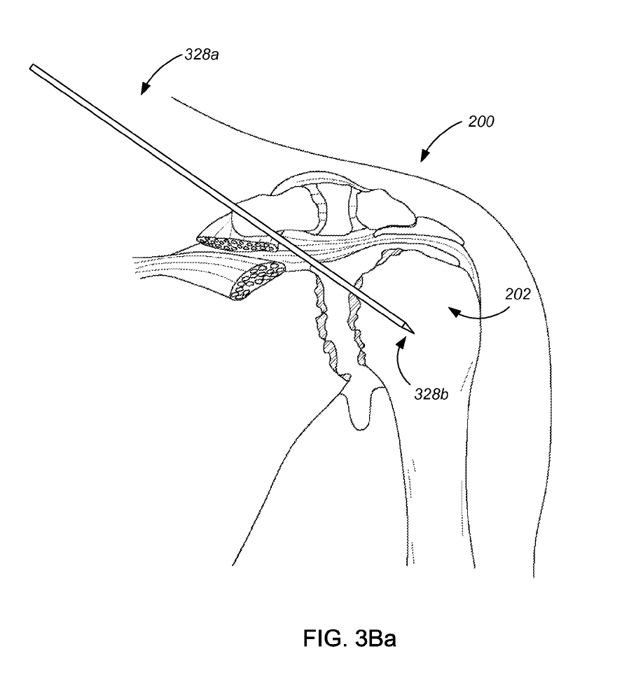

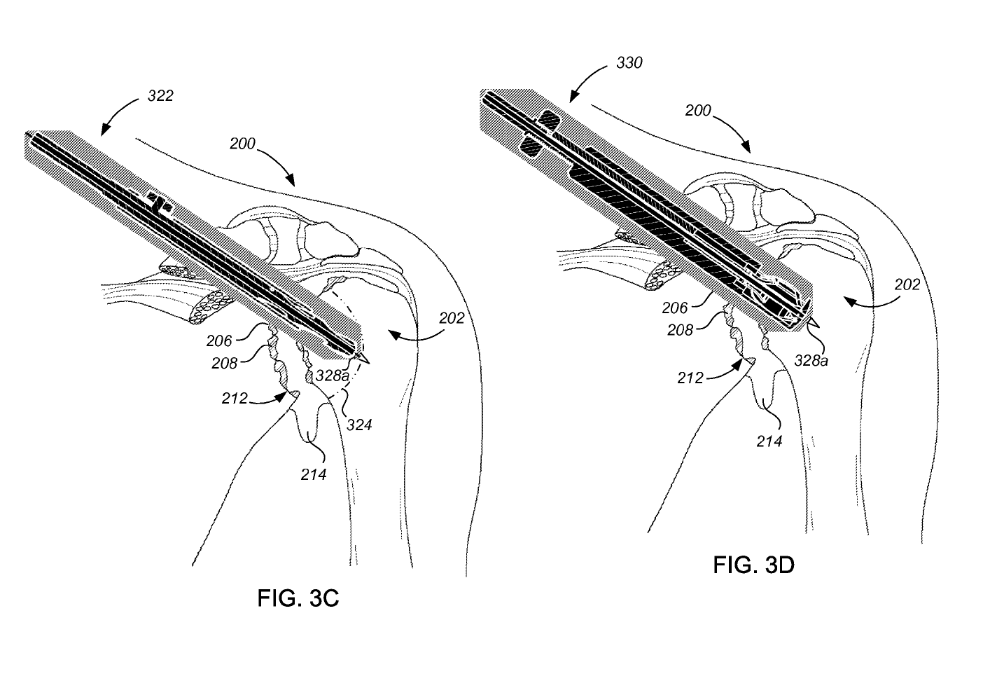

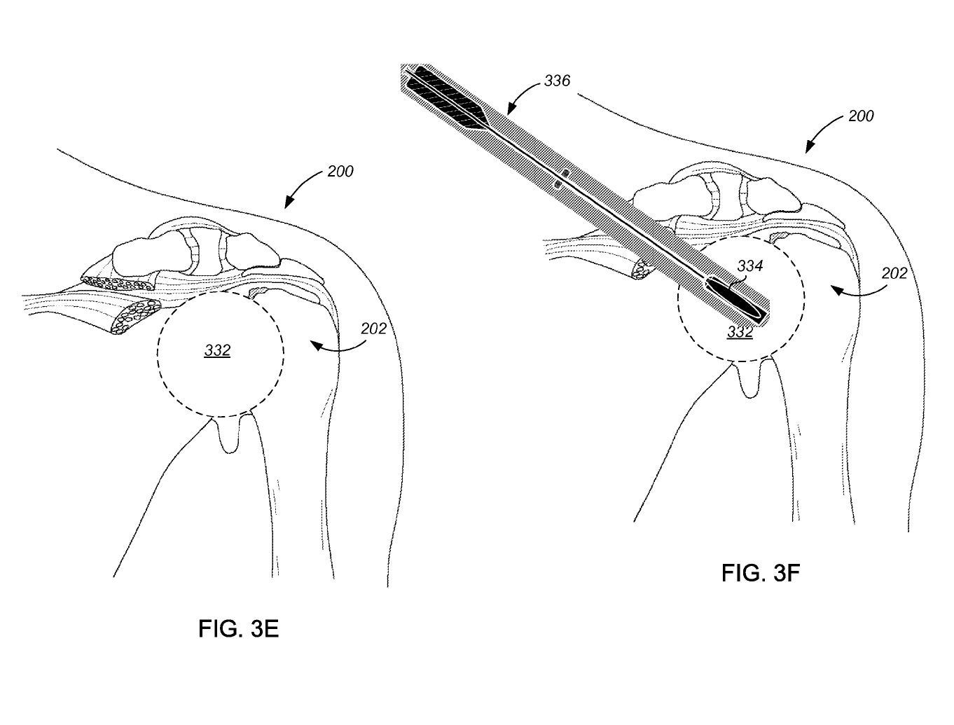

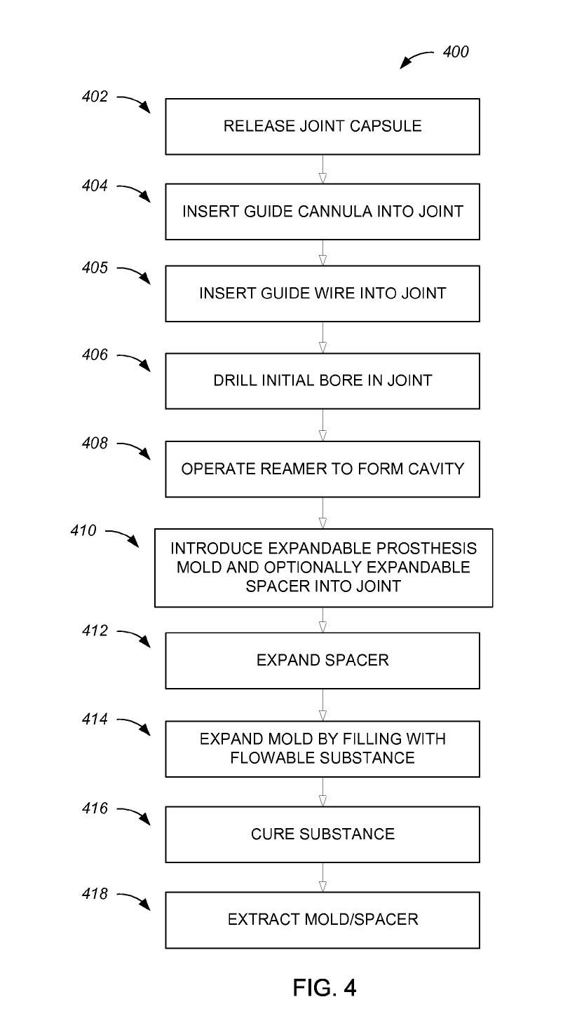

Reference is now made to FIGS. 3A-B which schematically illustrate damaged joint 200 and a first, optional element of a surgical kit, during at least a stage of the present surgical method for in-situ formation of a joint replacement prosthesis (the method also referred to as the “procedure”). Intermittent reference is also made to FIG. 4, which shows a flow chart 400 of the method.

Access to damaged joint 200 during the surgical method may be initialized through one or more incisions through the skin and the tissue covering the joint. Optionally, a small incision is formed, with a scalpel. Optionally, the incision is straight. The incision may be within the range of 4 to 10 millimeteres in size. The incision may be up to 15 mm long or more. Following incision, one or more working cannulas, as known in the art, may be applied, to facilitate straightforward access to the joint through the soft tissues envelope.

It is to be understood that the one or more incisions are small in comparison to the diameter of the prosthesis to be formed, rendering the method minimally-invasive according to accepted medical standards.

Optionally, a first incision is made for creating a portal for viewing, namely, for insertion of the arthro scope. One or more additional incisions may be formed to create one or more working portals for surgical instruments and substances of the method. Portals may be swapped during the procedure, if required, such that a viewing portal becomes a working portal, and vice versa.

In a step 402 (FIG. 4) of the method, arthroscopic release of a joint capsule 214 of damaged joint 200 may be performed (not shown in the figures), optionally using one or more known techniques such as diathermy, usage of an RF (Radio Frequency) wand, mechanical arthroscopic scissors and/or punches. Optionally, osteophytes are resected from the joint surface, on one or both joint surfaces.

Then, in a step 404, a guide cannula, such as guide cannula 328, may be inserted into damaged joint 200 through the incision, as FIG. 3A shows.

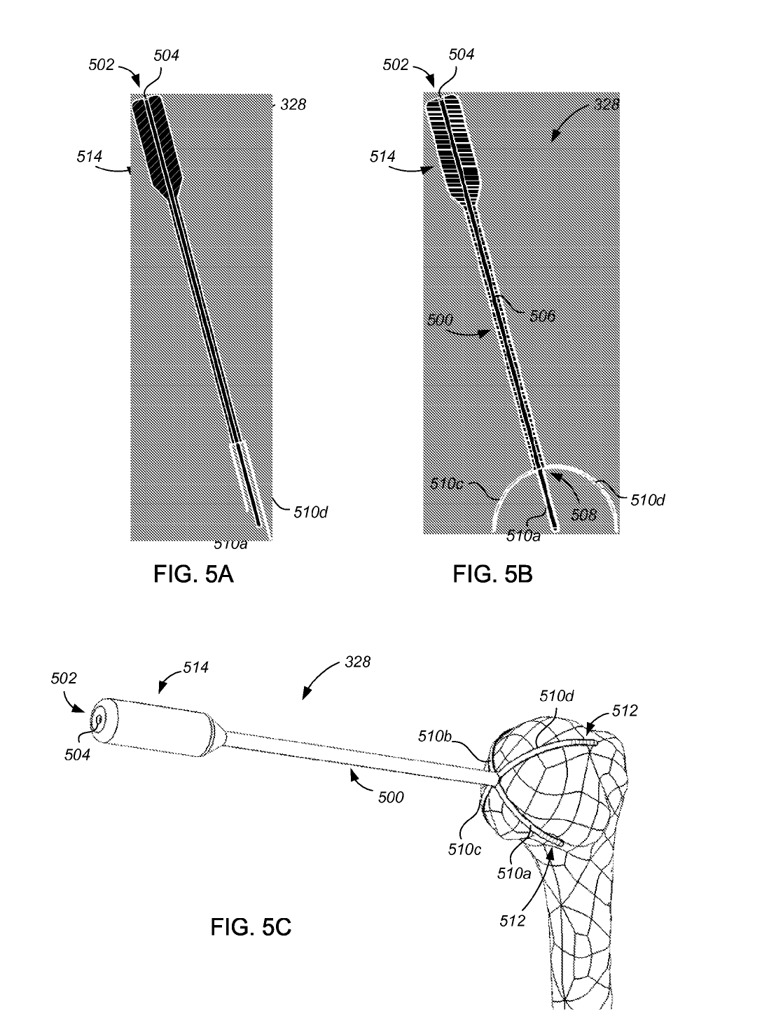

Reference is now made to FIGS. 5A-C, which show guide cannula 328 in more detail, from cross-sectional and perspective views. FIG. 5A shows guide cannula 328 in its collapsed form, while FIGS. 5B-C show it in its expanded form. Guide cannula 328 may serve to guide insertion of a central guide wire that will maintain an essentially constant relative orientation of various cannulated instruments inserted into damaged joint 200 (FIGS. 3A-B), over the guide wire, during the procedure. A proximal end 502 of a shank 500 of guide cannula 328 may include a proximal opening 504 for insertion of the central guide wire, the channel extending inside the shank up to a distal end 508 of the guide cannula. Shank 500 may include a thicker area near proximal end 502, serving as a grip 514.

It is to be understood that the terms ‘central guide wire’ and ‘guide wire’, as used herein, are interchangeable. These terms refer to a wire, optionally metallic, over which instruments, including cannulated instruments, are introduced to the joint at a desired site. However, the guide wire may guide non-cannulated tools, using alternative mechanisms, such as magnetic attraction. Optionally, the diameter of the guide wire is about 2 millimeters.

At or near its distal end 508, guide cannula 328 may include an expandable securing element, such as, for example, arms 510. The securing element serves to secure distal end 508 to humerus head 202, shown here in perspective, throughout at least part of the procedure. Arms 510, shown here only as one example of a securing element, may each be arc-shaped, with a radius of curvature matching or close to that of humerus head 202. Arms 510 may grip and/or circumferentially engage a sphere, an ellipsoid or any oval shaped figure. Guide cannula 328 may be provided with arms 510 of different sizes, to fit different patients. Optionally, graduation 512 is present on arms 510a-d, such that their position over humerus head 202 may be viewed by the surgeon and used for adjusting the position of the guide cannula, as necessary. The distance between graduation 512a on arm 510a and a neck of humerus head 202 may be used to determine the desired position of the guide cannula which in turn assists positioning the guide wire in said desired position. Further optionally, arms 510a-d are shaped and positioned such that they define a part of a sphere or an ellipsoid, so that shank 500 connects to the arms at a location indented to a central axis of the sphere or the ellipsoid. This may be achieved, for example, by providing two opposing arms 510a-b having essentially equal lengths, and two opposing arms 510c-d whose lengths are different.

Nonetheless, a different securing element (not shown) may be provided, for securing a guide cannula to a location other than the humerus head, in case that also a portion of the glenoid is to be resected. A distal opening 514 is provided at distal end 508, facilitating insertion of the central guide wire to humerus head 202 or a different part of damaged joint 200.

The securing element, be it arms 510 or a different element, may be introduced into damaged joint 200 when collapsed, as FIG. 5A shows, to enable insertion through a relatively small incision. When inside the joint, an expansion mechanism (not shown) may be triggered, to expand the securing element to its final measurements.

Returning to FIGS. 3A-B, guide cannula 328 is optionally placed over the joint surface and is used for directing the central guide wire into damaged joint 200 at a certain, optionally, predetermined, angle in relation to a neck of humerus head 202, the angle optionally matching the indentation of arms 510 in relation to shank 500. The neck is often referred to as the part of the humerus head which is situated between its spheroidal articular surface (shown more clearly at 102A in FIG. 1) and its elongated deltoid ridge (102B in FIG. 1). A virtual line surrounding the circumference of the neck is shown at 202C in FIG. 3A. A central axis of the neck, which is perpendicular to line 202C, is shown at 202D. An angle 202E between central axis 202D and guide cannula 328 is optionally between 0-10, 10-20, 20-30, 30-40 or 40-50 degrees. For example, angle 202E may be approximately 15 degrees.

Optionally, the choice of angle may assist to reduce the greater tuberosity of the humerus relative to the centre of rotation. This effect may improve the function of the deltoid muscles and the rotator cuff and may prevent impingement of the greater tuberosity on the acromion in elevation of the arm.

Reference is now made to FIG. 3Ba, which shows an exemplary guide wire 328a following its insertion, through guide cannula 328 of FIG. 3B, into humerus head 202 of damaged joint 200. The insertion of guide wire 328a may be performed in a step 405 of the method (FIG. 4). Guide wire 328a may include a relatively sharp point 328b, enabling its penetration into the bone by way of rapid spinning using a suitable spindle. Sharp point 328b may be conical, multi-faceted or the like. Optionally, sharp point 328b is threaded.

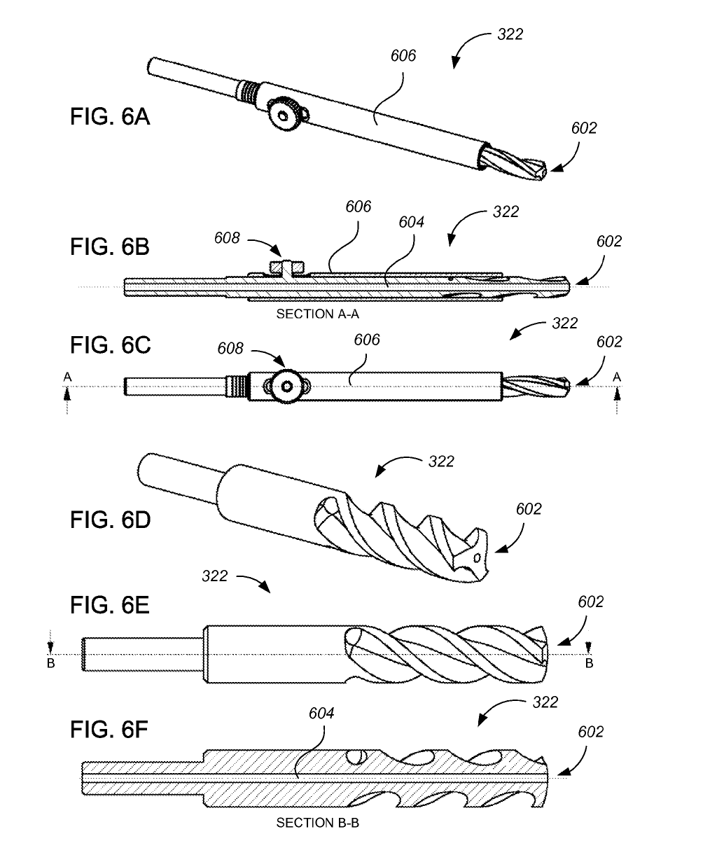

In a step 406 (FIG. 4) of the method, a starter drill 322 may be used to drill an initial bore, as shown in FIG. 3C, to which reference is now made, along with reference to FIGS. 6A-F, which show starter drill 322 in perspective, side and cross-sectional views. Starter drill 322 may be used in a cannulated manner, threaded over guide wire 328a into damaged joint 200.

Starter drill 322 may be a twist-type drill, optionally metallic, having a diameter of, for example, about 10 millimeters, and optionally ending with a convex surface 602. Starter drill 322 may include a guide channel 604 along its length, enabling it to be threaded on a guide wire (not shown). The diameter of a guide channel 604 is within a range that is appropriate for enclosing a guide wire 328a.

Optionally, starter drill 322 includes an adjustable stopper, such as stopper 606, allowing a surgeon to preset a desired drilling depth; upon reaching that depth, stopper 606 comes in contact with one or more of the articular surfaces and prevents further drilling Adjustability of stopper 606 may be enabled, for example, by an adjustment wheel 608 being configured, when rotated in one direction, to press onto and secure starter drill 322 in relation to the stopper, and, when rotated in the other direction, release the starter drill and allow its adjustment. Depth graduation (not shown) may be inscribed and/or printed on starter drill 322 and/or its stopper 606, allowing precise depth adjustment.

Following the drilling of step 406 (FIG. 4), starter drill 322 is removed from joint 200. Then, in a step 408, a convex, expandable reamer, such as reamer 330 shown in FIG. 3D, may be introduced into and operate on damaged joint 200. Reamer 330 may be configured to form the ellipsoidal cavity between the two bones. When first introduced into joint 200, reamer 330 may be in a collapsed form, as shown in FIG. 3D. The diameter of a reamer 330 in a collapsed form is optionally about 10 millimeter, for a joint in a human shoulder Similar or different diameters may be suitable for the human shoulder joint or different joints. Upon reaching a desired location within joint 200, an expansion mechanism may be triggered, to expand a tip portion of reamer 330. Upon expansion, the diameter of the reamer is enlarged, and may reach a diameter of approximately 36-40 millimeters or more for a human shoulder, or the same or different diameter for a different joint.

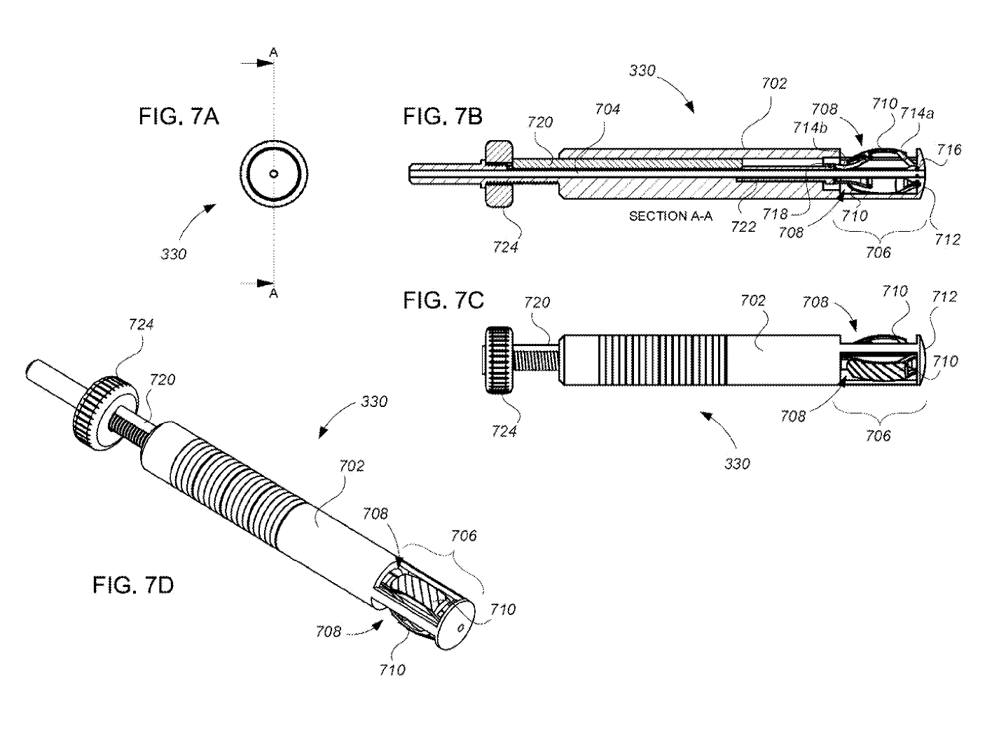

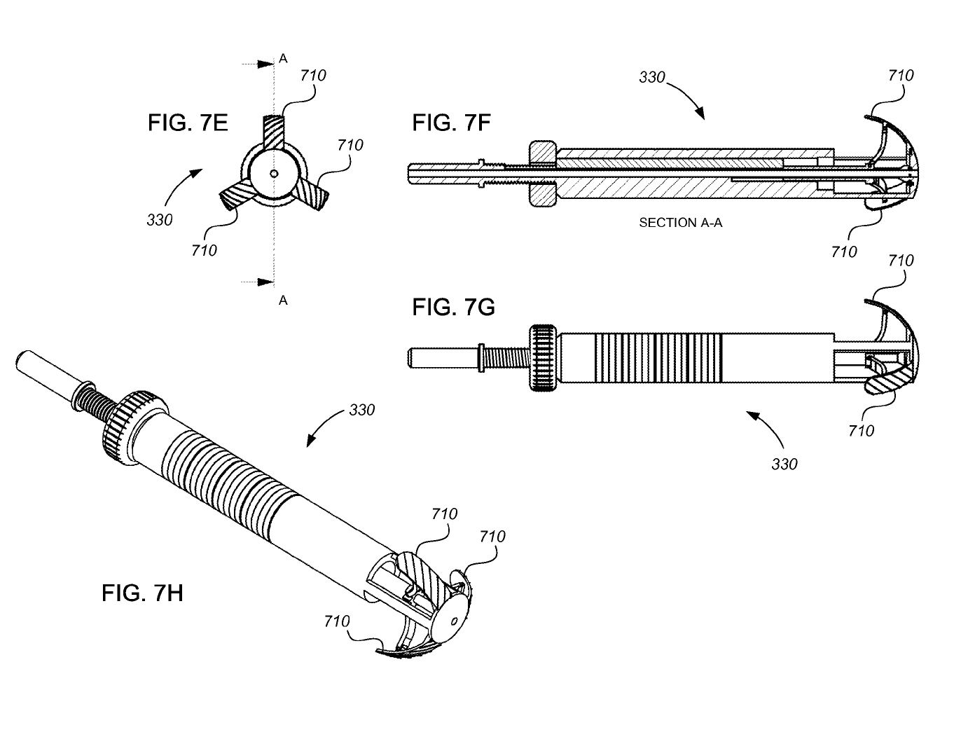

Reference is now made to FIGS. 7A-D, which show exemplary reamer 330 in its collapsed form in more detail, from front, cross-sectional, side and isometric views, respectively. Reamer 330 may include an elongated tubular housing 702, having a diameter suitable for introduction through the small skin incision.. (FIGS. 3C-D). An internal guide channel 704 may extend inside housing 702 along its length, enabling its threading on guide wire 328a (FIG. 3D).

A tip portion 706 of reamer 330 may include openings, for example three openings 708, enabling the expansion of one or more reaming blades, such as three reaming blades 710 (in some of the figures, only two openings and two blades are visible), outside housing 702. An alternative reamer (not shown) may include one or more reaming blades which protrude forwardly out of a housing, thereby requiring no openings in the housing for their expansion.

Blades 710 may each be arc-shaped, such that, when expanded, they form a curved surface together with a convex distal end 712 which bridges them. FIGS. 7E-H show reamer 330 with blades 710 expanded. The curved surface formed may match the diameter and/or shape of the desired ellipsoidal cavity to be formed. One or more of blades 710 may include diagonal stripes protruding from its external surface, and serving to enhance shaving of joint matter, such as bone, cartilage and/or the like.

One or more of blades 710 may be mounted onto two blade arms, a front blade arm 714a and a rear blade arm 714b. Front blade arms 714a may be connected to housing using pivots 716, and rear blade arms 714b may be connected to an expansion triggering mechanism using pivots 718. The expansion triggering mechanism enables the surgeon to gradually expand blades 710 when the reamer is properly inserted into position. The expansion triggering mechanism may be based on, for example, a triggering rod 720 extending from a proximal area of reamer 330 and along at least a portion of the length of housing 702. Triggering rod 720 may be contacting, further distally in housing 720, a triggering cylinder 722, which, in turn, is connected to pivots 718. A triggering wheel 724 may be threaded around housing 702 at the proximal area. When the surgeon desires to expand blades 710, she may turn and thread triggering wheel 724 towards the distal area of reamer 330, making the triggering wheel push triggering rod 720, which triggers cylinder 722, rear blade arms 714b and finally blades 710, causing the latter to expand. A motor (not shown) may be used to rotate reamer 330 so as to create the desired ellipsoidal cavity. The motor may be started, initially, when reamer 330 is still in its collapsed form. Then, while reamer 330 rotates, triggering wheel 724 may be gradually threaded towards the distal area of the reamer, thereby gradually expanding the formed cavity. In FIGS. 7E-H, triggering wheel 724 is shown fully threaded in the distal direction, and blades 710, accordingly, are fully expanded. Optionally, during at least a portion of the reaming, a net-like basket and/or a grater-like reamer may be used, to fine-smooth the internal surface of the formed cavity. Fine reaming is intended for obtaining a perfectly smooth cavity.

The term “net-like basket” as used herein refers to a perforated sphere, wherein the perforation is in the range of micrometers. This sphere is used as a fine file, and its use results in a smooth surface (typically, less than 20 microns of average surface roughness).

In order to form an ellipsoidal cavity, drilling and/or reaming, according to the above discussions, may be done at one or more different angles of approach (not shown), optionally through one or more additional incisions, such that blades 710 of reamer 330 can reach essentially the entire inner surface of the desired cavity. For example, drilling and/or reaming may be done to resect also part of the glenoid, if the glenoid and/or its cartilage are also damaged, so that the resulting cavity extends between the interfacing bones and within these bones themselves. FIG. 3E illustrates such an exemplary cavity 332. As a result of the reaming, the internal surface of cavity 332 may be exceptionally smooth, so as to facilitate smooth joint motion when the patient recovers.

Optionally, an expandable impactor (not shown), similar to reamer 330 but having an essentially complete hemispherical and smooth body instead of blades 710, may be used in addition to or instead of use of the reamer, to enhance the smoothing of the internal surface of cavity 332.

The term “impactor”, as used herein, may refer to an instrument which is capable of minimizing the average surface roughness. Upon impinging on the cavity's surface, the impactor causes mechanic deformation of the cavity's surface, to essentially the same geometry of the impactor itself. It compacts the cancellous bone in the concave internal surface of the formed cavity.

Optionally, the hemispherical surface of the expandable impactor is of essentially the same size and shape as the curved surface created by blades 710 of reamer 330 when expanded, but is smooth on its convex external surface. Thus, upon expansion, the expandable part may be shaped essentially as a hemisphere or a section of a sphere.

In a step 410 (FIG. 4), once an ellipsoidal cavity of a desired shape, size and location has been formed, forming of a joint replacement prosthesis may commence. First, however, guide wire 328a (FIG. 3D) may be removed from the joint.

With reference to FIG. 3F, an expandable prosthesis mold, such as mold 334, may then be introduced into cavity 332 (FIG. 3E) in a collapsed form. Optionally, mold 334 is an inflatable mold. Mold 334 may be made of a material being both flexible, so that it may be collapsed and expanded, as well as being non-stretchable beyond a certain final size, so that the size of the resulting prosthesis may be pre-determined by selection of a mold of a certain final size; this may obviate the need to precisely control inflation pressure during the procedure, since the mold will not be able to expand beyond that known size. A suitable material may be, for example, PEBAX (Polyether block amide), various Nylon blends, PET (Polyethylene terephtalate), Nylon (synthetic polymers known generically as polyamides) and/or the like. In addition, mold 334 may exhibit a highly smooth internal surface, without stitches, plastic injection marks and/or the like. This may ensure that the resulting prosthesis has a perfectly smooth external surface.

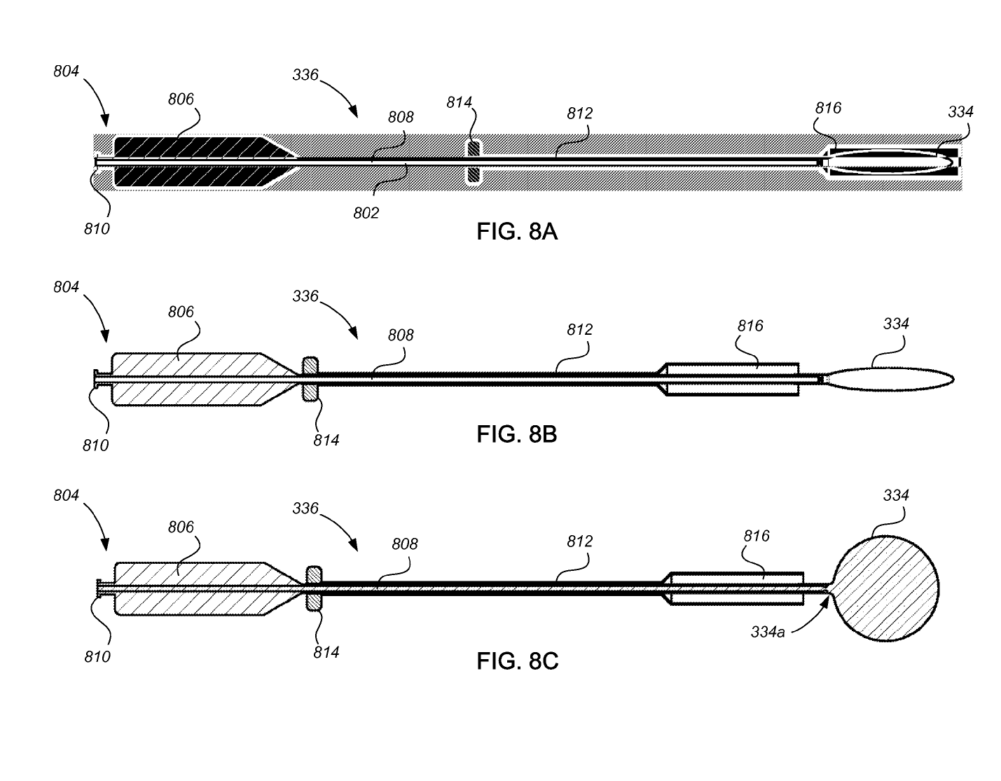

Optionally, an insertion instrument, such as insertion instrument 336, may be used for the introduction. Reference is now made to FIGS. 8A-C, which show cross-sectional views of mold 334 and insertion instrument 336 in more detail. Insertion instrument 336 may include an elongated tubular shaft 802 which is optionally thicker near its proximal end 804, forming a grip 806. A working channel 808 may extend internally inside shaft 802. An input port 810 may be positioned at a proximal end of working channel 808.

Insertion instrument 336 may further include an extendible liner, such as liner 812, being a hollow cylindrical instrument mountable around shaft 802, having a handle 814 and optionally a storage chamber 816. Before introduction into the joint, mold 334 may be stored inside storage chamber 816, which protects it during the insertion. Mold 334 may include a narrow neck (not shown), extending inside shaft 802 optionally up to its proximal end or even further, thereby enabling filling of the mold. Alternatively, a separate inflation tube (not shown) may extend inside working channel 808 and connect to mold. Mold 334 may be positioned inside cavity 332 (FIG. 3F) such that, when the mold is fully expanded, the mold is either at the center of cavity or indented from the center.

When mold 334 and storage chamber 816 have been inserted into cavity 332 (FIG. 3F), handle 814 may be pulled by the surgeon, thereby pulling the entire liner 812 backwards and exposing the mold.

Optionally, mold 334 is provided inside an expandable spacer (not shown in this figure), configured to maintain cavity 332 (FIG. 3F) between the two interfacing bones at least during the formation of the prosthesis. The spacer, similar to mold 334, may have a final size when expanded, the final size optionally matching that of cavity 332 (FIG. 3F). Upon expansion of the spacer, it prevents the interfacing bones and/or other parts of the joint from collapsing and/or otherwise interfering with the formation of the prosthesis inside mold 334. According to other embodiments, mold 334 itself may be configured to expand such that it prevents the interfacing bones and/or other parts of the joint from collapsing and/or otherwise interfering with the formation of the prosthesis inside mold 334, thus obviating the need for a spacer. Each possibility represents a separate embodiment of the present invention. Mold 334 may be configured to expand under high pressure, thus maintaining cavity 332 between the two interfacing bones at least during the formation of the prosthesis and obviating the use of a spacer.

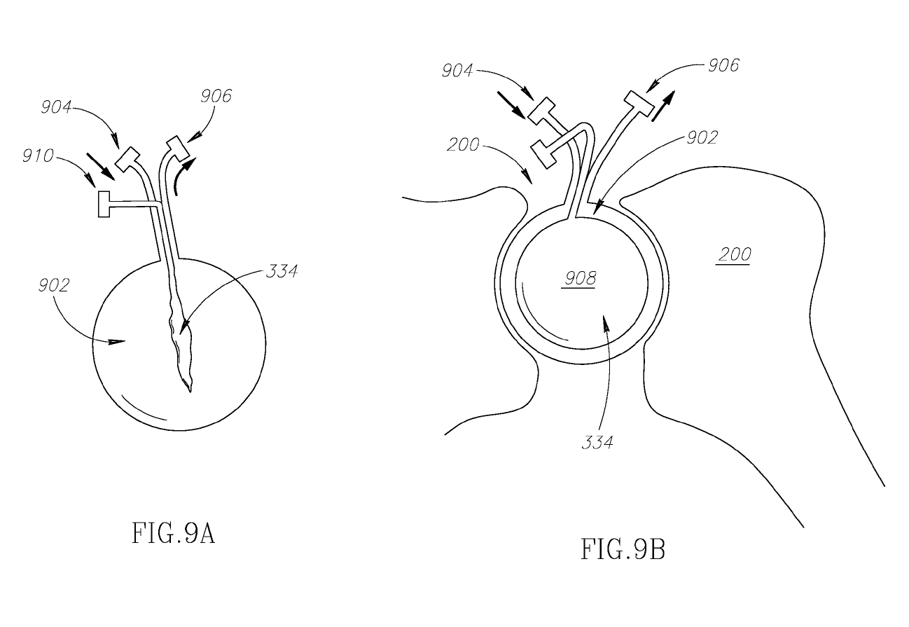

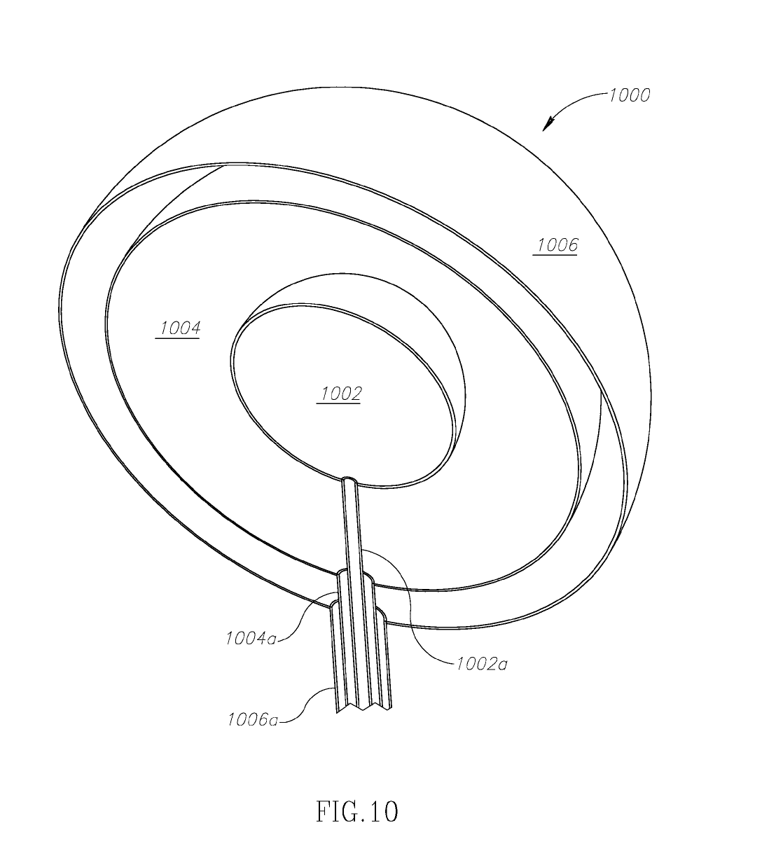

Reference is now made to FIGS. 9A-B, which show cross-sectional views of mold 334 and an optional spacer 902 containing the mold. Spacer 902 is shown already expanded, following its introduction into damaged joint 200 when collapsed over the collapsed mold 334, the two constituting, essentially, a double-lumen expandable apparatus. Optionally, the joint introduction of the two is performed using an insertion instrument, such as insertion instrument 336 of FIGS. 8A-C.

FIG. 9A shows mold 334 in its collapsed form inside the expanded spacer 902. For simplicity of presentation, necks of mold 334 and spacer 902 are shown schematically, without reference to an insertion instrument, pumping system and/or the like. In a step 412 (FIG. 4), spacer 902 is expanded. Optionally, the expansion is by pumping air or fluid into a filling neck 904 of spacer 902, while keeping an emptying neck 906 of the spacer closed. It is to be noted that a spacer according to the present invention may have the shape of spacer 902 or other shapes, such as forceps, expandable arms and the like, so as long as the spacer is configured to maintain the cavity between the two interfacing bones at least during the formation of the prosthesis.

Then, in a step 414 (FIG. 4), mold 334 is expanded, by filling it with a flowable, curable substance, through its filling neck 910, to form a prosthesis 908 inside it. Optionally, solids (not shown) may be intermixed with the substance, for purposes such as reducing the weight of prosthesis 908, structurally enforcing the prosthesis and/or the like. For example, hollow globules may be intermixed with the substance.

A final size of mold 334 may be such that a gap is maintained between the outer surface of the mold and the inner surface of spacer 902. For example, the diameter of mold 334 may be a few millimeters to about 1 centimeter smaller than that of spacer 902, leaving a gap of a few millimeters, for example, 5 millimeters between the two. A priming step may be performed at the beginning of the filling. While filling the prosthesis mold, the pressure in the outer spacer is preserved. This may be achieved by allowing escape of the fluid or air through the output neck.

In a step 416 (FIG. 4), following the filling of mold 334, curing of the substance may take place, for example using an ultraviolet (UV) illuminator (not shown), introduced arthroscopically. Alternatively, if an Epoxy is used, curing will occur without external intervention. Suitable materials include, for example, EPO-TEK UVO—114 (UV curable, command cure), EPO-TEK 715 and/or Stryker medical's Simplex P SpeedSet among others.

The curing process may be exothermic. If so, an arthroscopic fluid, such as saline, may be used to flush the surroundings of mold 334 during the curing, so that no tissue is substantially affected by the heat. Following the curing, prosthesis 908 is rigid, ellipsoidal and smooth.

Optionally, prosthesis 908 may be enlarged by forming one or more additional prosthetic layers over it. This may be performed, for example, by providing multiple expandable molds of gradual sizes one inside the other, and filling and curing them sequentially. If this option is employed, a spacer may or may not be used. One or more of the expandable molds may be isomorphic and/or axisymmetrical at least after a prosthesis and/or a prosthetic layer(s) has been formed inside them. Alternatively, one or more of the expandable molds may not be isomorphic and/or axisymmetrical. In some alternatives, axisymmetricality of the prosthesis and/or a prosthetic layer(s) is of less importance, since when a layer covers its predecessor, the lack of axisymmetricality is not exhibited.

Reference is now made to FIG. 10, which shows, in perspective, an example of three molds of gradual sizes provided one inside the other. An internal mold 1002 is filled first, through its neck or inflation tube 1002A, to form a first prosthesis. It is then cured. Next, an intermediate mold 1004 is filled, through its neck or inflation tube 1004A, to form a prosthetic layer over the previous prosthesis. Curing follows the filling. Finally, an external mold 1006 is filled, through its neck or inflation tube 1006A, to form and cure another prosthetic layer over the previous prosthetic layer.

Optional advantages of forming a large prosthesis gradually, rather than creating a large prosthesis at once using one mold, include, for example, reduced curing time, lower temperature (if curing is an exothermic reaction), better-controlled hardening, higher confidence, reduced bubble formation and/or reduced imperfections. The reduced curing time leads to improved curing, since less material is involved in each curing step and less outgassing is caused. Incremental buildup of the implant increases strength for compression and eliminates stress cracking, micro cracks and other material imperfections.

When multiple molds are used to form a prosthesis and a prosthetic layer(s) sequentially, one or more of the molds may not be removed from the joint prior to forming the next prosthetic layer over them. If a mold is to remain in place, it may be manufactured of a material or a combination of materials suitable for staying inside the cured substance permanently. One example of such a material is THV (Terpolymer of Tetrafluoroethylene, Hexafluoropropylene and Vinylidene fluoride). THV is an extremely flexible fluoropolymer, having excellent optical clarity. Combined with the traditional chemical and environmental resistance of fluoropolymers, THV may be a suitable material for forming one or more of the molds. THV provides, for example, excellent permeation, good UV transmittance and more.

Either if a single mold or multiple molds are used, it may be desired to extract one or more of them following the curing, and optionally also the spacer. In a step 418 (FIG. 4), extraction of a mold or a spacer may be performed.

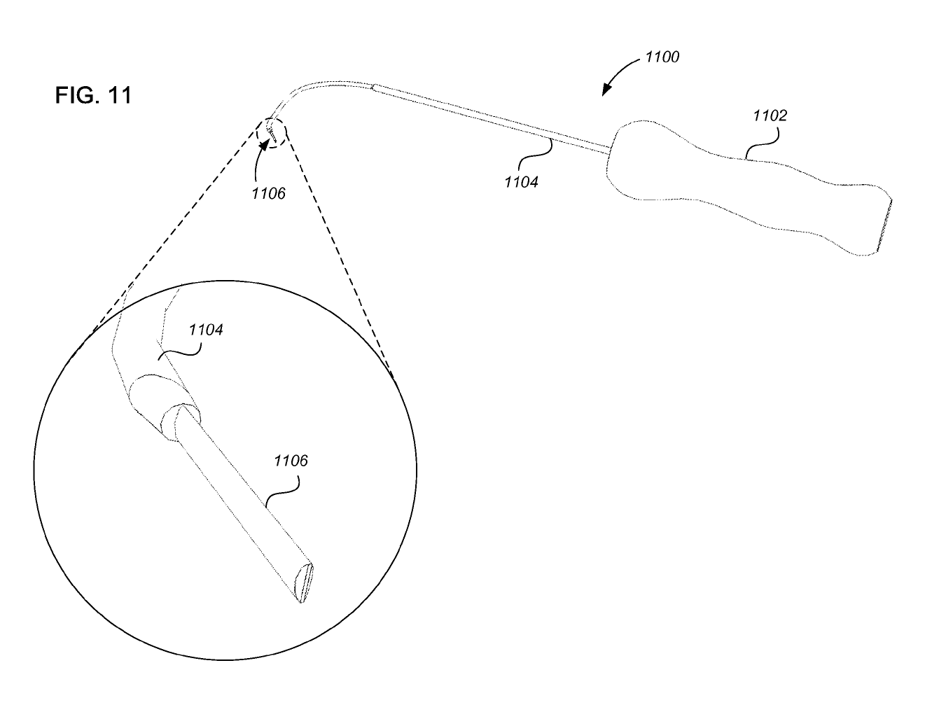

One or more instruments may be used for extraction, such as the instruments shown in FIGS. 11-15. A first example is shown in FIG. 11, to which reference is now made. An extraction instrument 1100 may include a handle 1102, to which an elongated, flexible arm 1104 is connected. Arm 1104 may be curved towards it end, so that it may be wrapped around the ellipsoidal mold once inside the joint. Arm 1104 may terminate with a blade 1106. To extract a mold or a spacer, arm 1104 is arthroscopically introduced into the joint, such that its curved area wraps around at least a portion of the mold or spacer. Blade 1106 may then contact the mold or spacer. When extraction instrument 1100 is pulled, blade 1106 may drag on the surface of the mold or the spacer, forming an elongated cut, whether to the full thickness of the mold/spacer or a part of it. Then, the torn mold or spacer may be pulled out of the joint through their necks or inflation tubes.

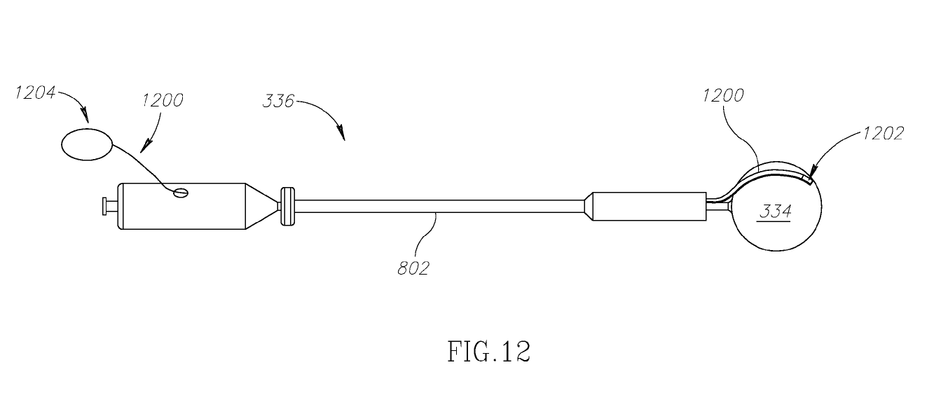

A second example of an extraction instrument is shown in FIG. 12. This extraction instrument may include an elongated, flexible strip 1200 threaded through an arthroscopic instrument, such as insertion instrument 336 of FIGS. 8A-C, while mold 334 is still held by the insertion instrument. As strip 1200 progresses inside shaft 802, it reaches mold 334 and contacts its outer surface. Further pushing of strip 1200 causes it to progress on the outer surface of mold 334, until it reaches desired position 1202. Pulling strip 1200, for example using handle 1204, enables ripping and/or pulling of mold 334. Each possibility represents a separate embodiment of the present invention.

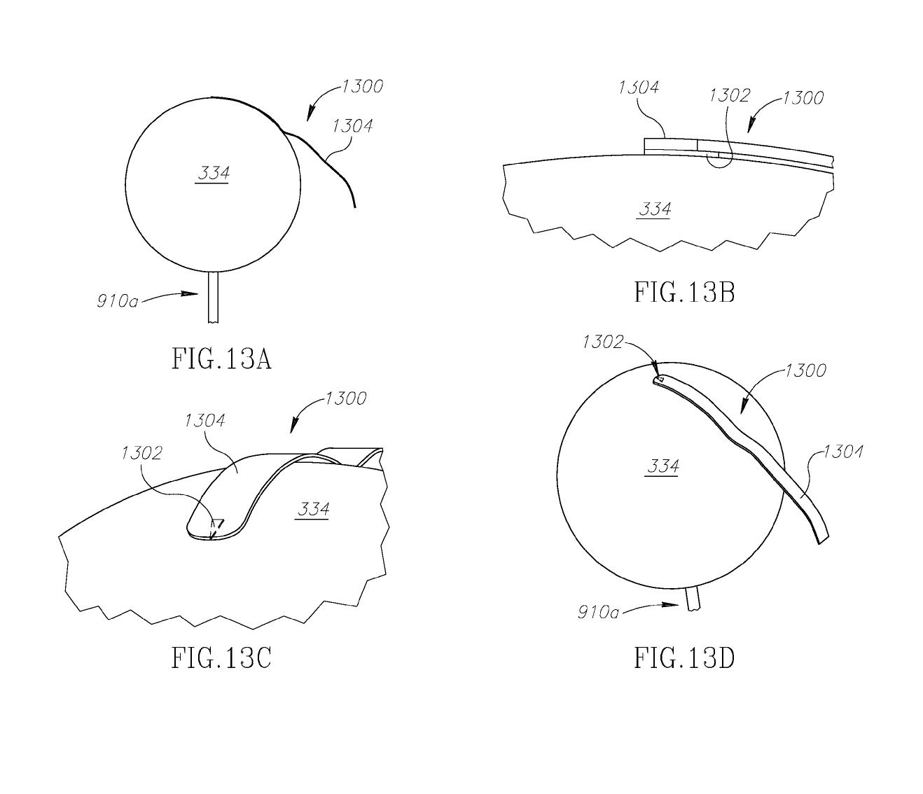

FIGS. 13A-D show a third example of an extraction instrument 1300 for mold 334 connected to neck 910a. A thin, triangular polymeric film 1302 may be affixed, using an adhesive, to the outer surface of the mold or spacer. Then, a flexible polymeric strip 1304 is introduced into the joint and adhered, at its edge, to film 1302. Strip 1300 is optionally inserted into the joint similar to the insertion of strip 1200 of FIG. 12. When strip 1304 is pulled, concentrated shear forces are created on the outer surface of the mold, at the point where one of film's 1302 sharp corners is located. If the mold is made of a polymer, these forces may cause plastic deformation of the mold, finally leading to its rupture. then, while strip 1304 continues to be pulled, an elongated crack is formed in mold, such that it may be easily extracted by pulling out through its neck or inflation tube.

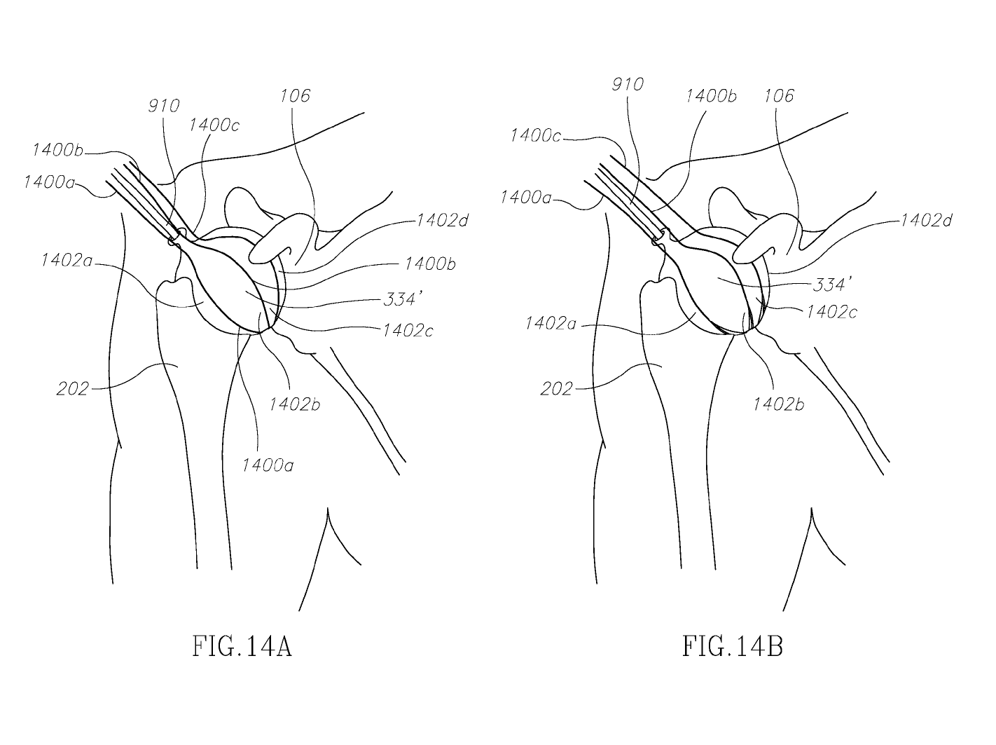

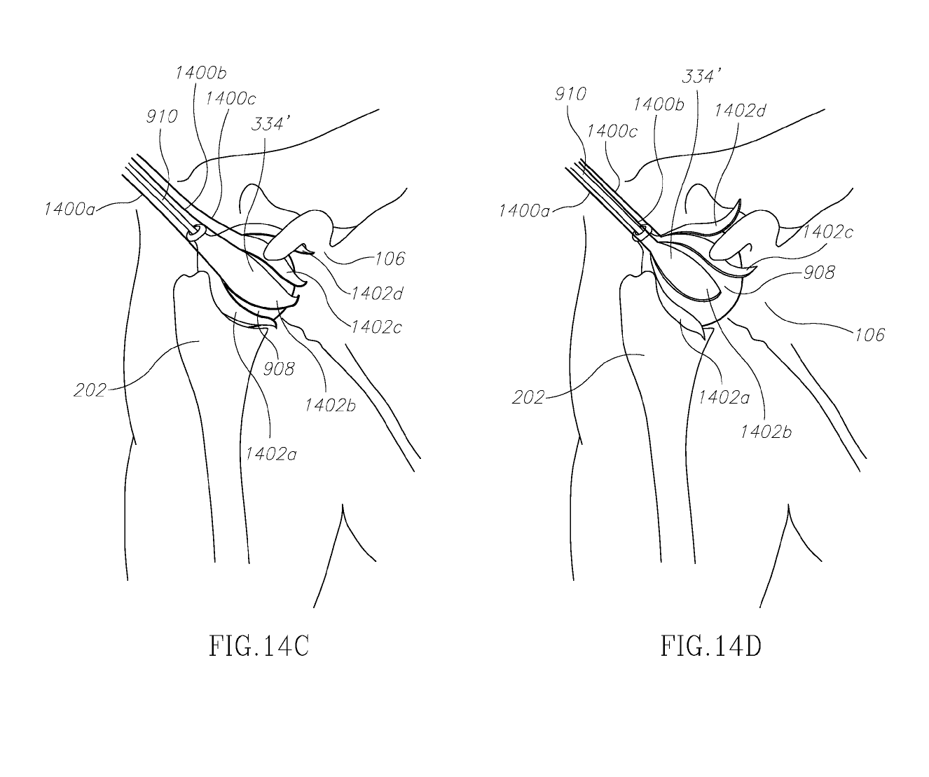

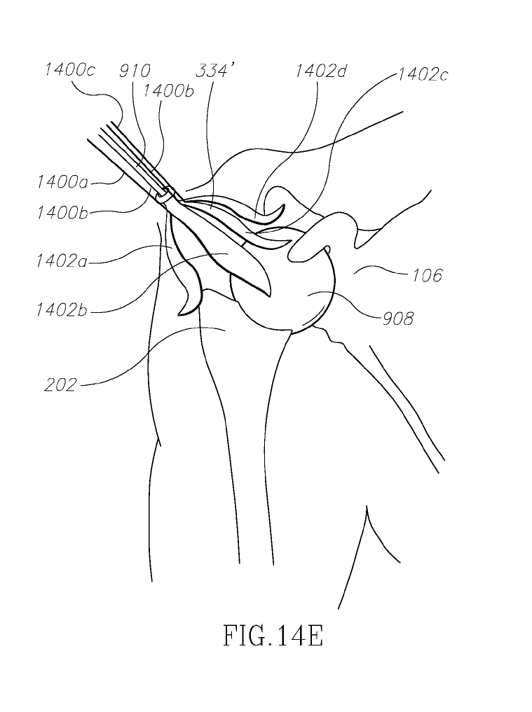

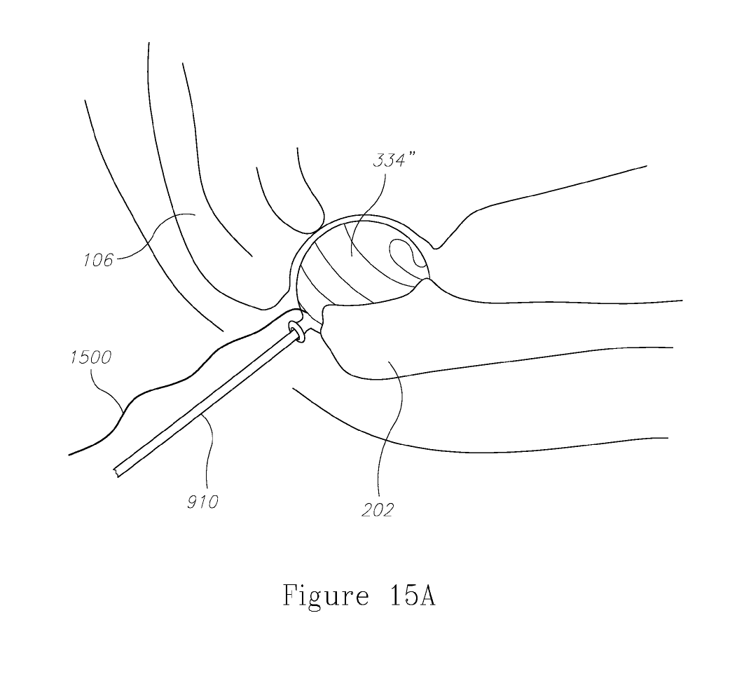

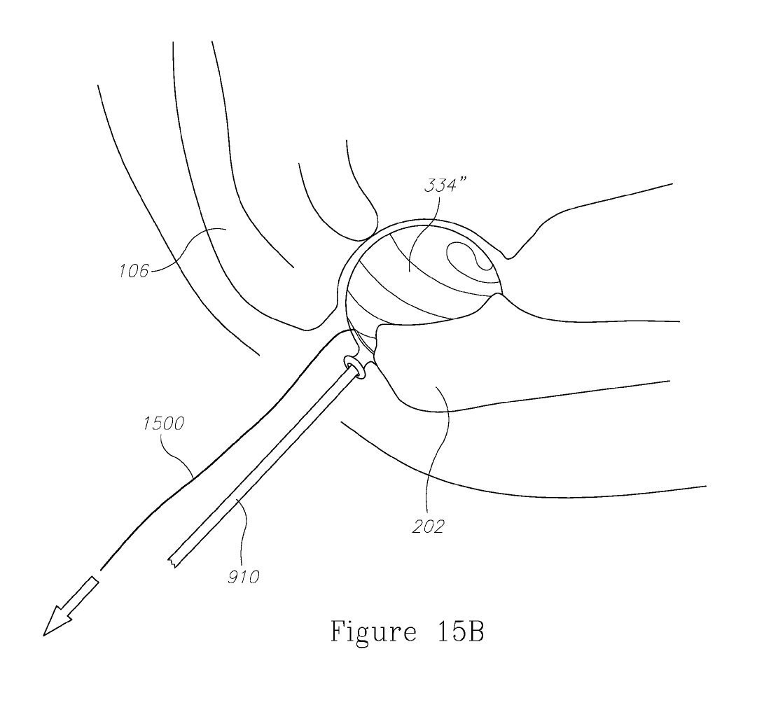

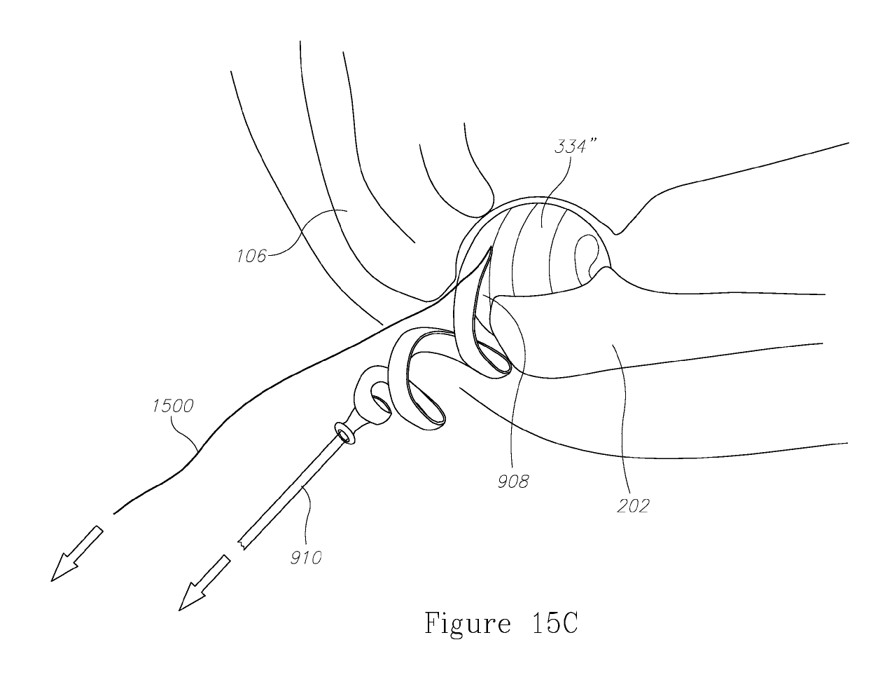

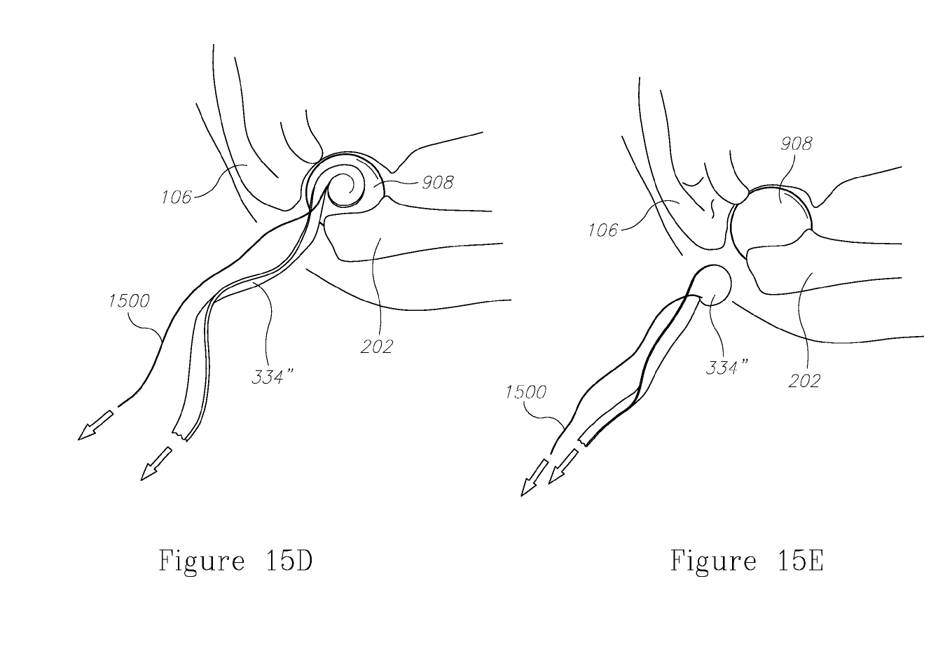

According to other examples, the mold and/or spacer may be extracted by providing them with one or more wires, at least partly attached to or embedded in their ellipsoidal surface and extending to their necks/inflation tubes. To extract them, the wires may be pulled, causing the tearing of the mold or spacer along the path of the wires and the pulling of the torn parts outside. FIGS. 14 and 15 depict exemplary extracting instruments using such wires.

A fourth example of an extraction instrument is shown in FIGS. 14A-E, showing mold 334′, situated between glenoid cavity 106 and humerus head 202, following filling of mold 334′ and formation of prosthesis 908. According to the example shown in FIGS. 14A-E, mold 334′ comprises wires 1400a-c attached to or embedded within the surface of mold 334′. Each possibility represents a separate embodiment of the present invention. Wires 1400a-c extend on or within the surface of mold 334′, from the apex of mold 334′ situated opposite the opening of neck 910 and in the direction of neck 910, possibly threading through shaft 802 of an arthroscopic instrument such as insertion instrument 336 of FIGS. 8A-C. According to some embodiments, the surface of a mold may comprise two or more wires, embedded in or attached to the surface, the wires roughly dividing the surface to at least 2 slices. Each possibility represents a separate embodiment of the present invention. Wires 1400a-c arc attached to or embedded in the surface of mold 334′ such that they roughly divide its surface to slices 1402a-d. Each possibility represents a separate embodiment of the present invention. According to some embodiments, slices 1402a-d may be of the same or different sizes. According to some embodiments, wires 1400a-c do not extend through the entire length of mold 334″s surface up to the connection point of neck 910 with mold 334′ but protrude from the surface before reaching the connection point, thus allowing the proximal part of mold 334′ to remain intact following pulling of wires 1400a-c. As used herein, the term “proximal” refers to the side closer to the care giver using the surgical kit and/or mold of the invention.

Pulling of the proximal part of wires 1400a-c is configured to induce ripping of the surface of mold 334′ along the path of wires 1400a-c. It is to be noted that ripping of mold 334′ by each wire of wires 1400a-c may be such that ripping occurs only at a single point at a time and not along the entire wire simultaneously. Ripping of mold 334′ at a single point at a time may require a lower ripping force, thus facilitating easier and/or faster ripping. Wires 1400a-c may be pulled through an arthroscopic instrument such as insertion instrument 336 of FIGS. 8A-C. FIG. 14A depicts mold 334′ prior to pulling of wires 1400a-c. FIG. 14B depicts mold 334′ once pulling of wires 1400a-c has started, thus ripping of the surface of mold 334′ begins at the apex of mold 334′ situated opposite the opening of neck 910. FIGS. 14C-D depict mold 334′ as the ripping of its surface continues with further pulling of wires 1400a-c, thus slices 1402a-d detach from prosthesis 908. FIG. 4E shows mold 334′ as it fully detached from prosthesis 908 following ripping of the mold's surface by wires 1400a-c. Following detachment of mold 334′ from prosthesis 908, mold 334′ is extracted from the subject, possibly through an arthroscopic instrument such as insertion instrument 336 of FIGS. 8A-C. Mold 334′ may be extracted by pulling on the proximal part of wires 1400a-c and/or by pulling neck 910 and/or by pulling a string directly connected to mold 334′ (not shown). Each possibility represents a separate embodiment of the present invention.

Wires 1400a-c or 1500, as depicted in FIGS. 14 and 15 may be rigid and/or strong enough to enable pulling them in order to rip and extract mold 334′ or 334″, respectively, but thin enough so that they do not cause bumping or blistering in the surface of mold 334′ or 334″, respectively, in a way that may affect the smoothness of prosthesis 908. Wires 1400a-c or 1500, as depicted in FIGS. 14 and 15 may be at least partly integrally formed with mold 334′ or 334″, respectively. Wires at least partly integrally formed with a mold, according to the present invention, may differ from the mold by properties such as, but not limited to, strength, rigidity, thickness, electric conductivity and the like.

According to some embodiments, mold 334′ is weakened prior to and/or during pulling of wires 1400a-c. Each possibility represents a separate embodiment of the present invention. Weakening of mold 334′ may be through exposure of mold 334′ to energy such as, but not limited to, heat energy, electrical energy, light energy, radio frequency, ultrasonic energy and the like, or any combination thereof. Each possibility represents a separate embodiment of the present invention. Exposure of mold 334′ to energy may induce heating and/or change in physical properties of mold 334′, thus leading to its weakening. Each possibility represents a separate embodiment of the present invention. Weakening of mold 334′ may facilitate easier and/or faster ripping of mold 334′ by wires 1400a-c, ultimately facilitating easier extraction of mold 334′ from the subject's body. According to some embodiments, weakening the surface of mold 334′ by electricity and the like may enable using wires 1400a-c that are thinner and/or less rigid than wires used without weakening mold 334′. Each possibility represents a separate embodiment of the present invention.

Exposure of mold 334′ to energy may be through the use of an energy source external to the subject's body, such as, but not limited to, an ultrasonic transducer, a radio frequency emitter and the like. An energy source external to the subject's body may transmit energy such as, but not limited to, ultrasonic energy or radio frequency to mold 334′. The external energy source may not have to be physically engaged with mold 334′ in order to transfer energy to the mold.

Alternatively, an energy source, such as, but not limited to, an electricity source, may physically engage with mold 334′ through wires, such as, but not limited to, wires 1400a-c. Accordingly, wires 1400a-c may optionally be made of a material such as, but not limited to, a metal, configured to transmit energy such as electricity. The proximal side of wires 1400a-c may optionally be connected to an external energy source, such as but not limited to, an electricity source. Alternatively, wires 1400a-c may be connected to an energy source which is inserted into the subject's body, possibly situated on an insertion instrument such as insertion instrument 336 of FIGS. 8A-C. The energy source may transfer energy, such as an electric current, through wires 1400a-c, thus heating the surface of mold 334′. The heating of the surface of mold 334′ may weaken the surface. According to some embodiments, at least two wires, such as wires 1400a-c, extending from mold 334′, are connected to an electricity source external to a the subject's body, such that wires 1400a-c and the electricity source form a closed electrical circuit. Alternatively, mold 334′ may be formed of an electricity conducting material such that wires 1400a-c, mold 334′ and the external electricity source form a closed electrical circuit. The electrical source may possibly be inserted into the subject's body and form a closed electrical circuit with wires 1400a-c and possibly with mold 334′. An electrical source inserted into the subject's body may be situated on an insertion instrument such as insertion instrument 336 of FIGS. 8A-C or possibly situated on mold 334′ itself or on a spacer surrounding mold 334′ (not shown).

A fifth example of an extraction instrument is shown in FIGS. 15A-E, showing mold 334″, situated between glenoid cavity 106 and humerus head 202, following filling of mold 334″ and formation of prosthesis 908. According to the example shown in FIGS. 15A-E, mold 334″ comprises a single wire 1500 attached to or embedded within the surface of mold 334″. Each possibility represents a separate embodiment of the present invention. Wire 1500 follows a spiral course on or within the surface of mold 334″, starting at the apex of mold 334″ situated opposite the opening of neck 910 and extending towards neck 910. The proximal part of wire 1500 extends towards neck 910 and may extend through an arthroscopic instrument such as insertion instrument 336 of FIGS. 8A-C. Pulling on the proximal part of wire 1500 is configured to result in spiral ripping of the surface of mold 334″ along the path of wire 1500, thus exposing prosthesis 908, as can be seen for example in FIGS. 15C-D. It is to be noted that ripping of mold 334″ by wire 1500 may be at a single point at a time and not along the entire length of wire 1500 simultaneously, thus possibly requiring a lower ripping force. A lower ripping force may enable faster and/or easier ripping of mold 334″. The ripping of mold 334″ by wire 1500 may start at the apex of mold 334″ situated opposite the opening of neck 910 and advance in the direction of neck 910, or, alternatively, start at a side of mold 334″ closer to neck 910 and advance in the opposite direction from neck 910 with pulling of wire 1500. Pulling on the proximal part of wire 1500 and/or pulling neck 910 and/or pulling a string directly connected to mold 334″ (not shown) results in extraction of mold 334″ from the subject's body, as can be seen in FIG. 15E.

As described for wires 1400a-c, wire 1500 may be made of a material configured to transmit energy to mold 334″. Similarly to mold 334′, mold 334″, may be weakened prior to and/or during pulling of wire 1500 through transmission of energy to mold 334″, such as, but not limited to, electrical energy and/or sound energy. The energy may be transmitted to mold 334″ through wire 1500 which is connected to an energy source external or internal to the subject's body. Alternatively, the energy source may transfer energy to mold 334″ without being physically connected to mold 334″.

Following the curing of the material inside the mold, a protrusion of cured substance may still remain in the area of the mold's neck or inflation tube (also its “input port”). Reference is now made to FIG. 8C, which shows the area where the protrusion 334A is formed. In an optional step of the method, a file, a cutter and/or the like may be used to remove the protrusion, so as to leave the mold completely smooth near its input port.

As an alternative to the in-situ formation of a joint replacement prosthesis such as prosthesis 908, using an expandable mold, such as mold 334, a different joint replacement prosthesis may be assembled inside the joint in a minimally-invasive manner. A joint replacement prosthesis assembly may be comprised of a plurality of parts, each having a small enough size which enables its introduction into a damaged joint in a minimally-invasive manner. The term “size” may refer to a measurement of the largest dimension of such part, which measurement dictates the size of an incision to made in the patient's skin and the size (for example diameter) of joint tissue which needs to be cleared away en route the site where the prosthesis is to be assembled. The parts may be introduced into the site individually, and assembled inside the site to form an ellipsoidal joint replacement prosthesis, which may have a smooth outer surface.

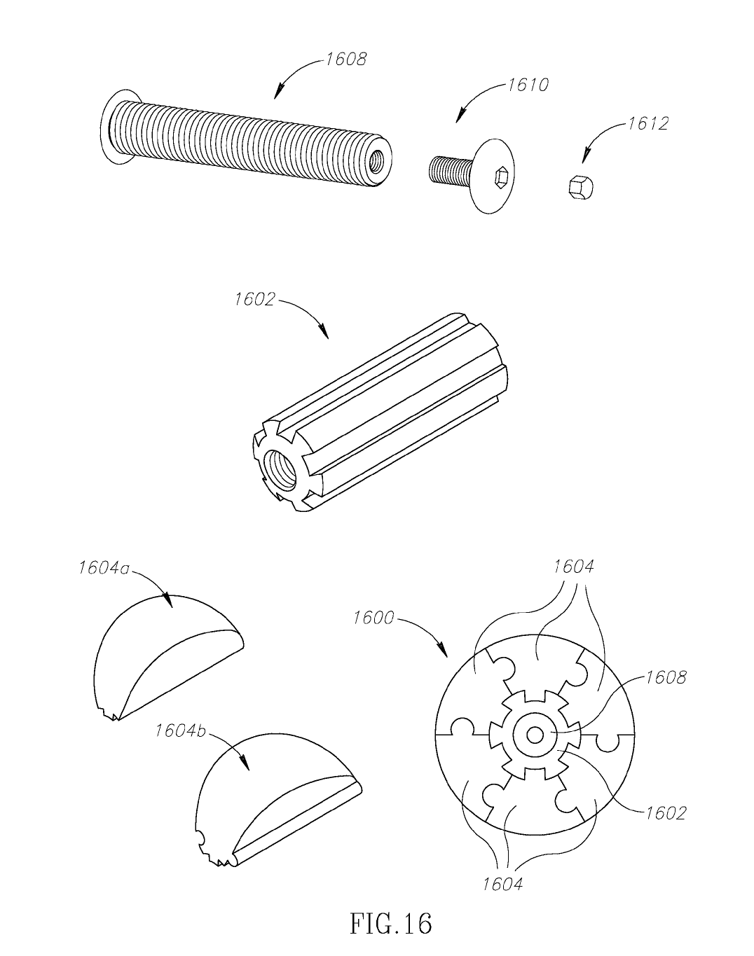

Reference is now made to FIG. 16, which shows an exemplary joint replacement prosthesis assembly (herein after “prosthesis assembly”) 1600 in a cross-sectional view, as well as the individual parts forming the prosthesis assembly.

Generally, prosthesis assembly 1600 may be assembled from a core structure (hereinafter “core”) 1602, having mounted thereon multiple slices, such as slices 1604, together forming the desired ellipsoidal shape—in this example a sphere. Six slices 1604 are shown in this figure, but any number of slices which is two or above is intended herein. The geometry of each of slices 1604 may include a bottom structure enabling its attachment to core 1602, and optionally a side structure enabling the slices to be attached to one another. Option A for slices 1604a includes only a bottom structure, while Option B for slices 1604b includes both a bottom structure and a side structure.

Prosthesis assembly 1600 may be introduced into and assembled inside the formed joint cavity as follows: Core 1602 and each of slices 1604 may be inserted into the cavity individually, such that only a relatively small incision and pathway are needed, similar to the rationale of mold 334 forming prosthesis 908 (FIGS. 9A-B). This facilitates the minimally-invasive nature of the procedure. Slices 1604 are then being slid onto core 1602, such that the bottom structures of the slices mount onto matching structures in the core. When all slices 1602 are mounted on core 1602, a securing bolt 1608 may be inserted into a central threaded bore of the core, and fastened using an opposite bolt 1610. This finally locks slices 1602 and core 1602 together, serving as a locking mechanism. Optionally, one or both of bolts 1608 and 1610 are hex-socket (also Allen) bolts. Bolts 1608 and 1610 may each have a cap being sufficiently large so that it covers at least the area where the bottom structures of slices 1604 and the matching structures in core 1602 meet. This way, the caps keep slices 1604 from sliding off core 1602. A bit, such as bit 1612, may be used to seal each hex socket, such that prosthesis assembly 1600, as a whole, has the smoothest possible external surface.

As an alternative to having securing bolt 1610 and core 1602 as separate pieces, they may be combined in a single core body (not shown). This core body will have one end with a rim preventing the slices from sliding off at that end, and a bore at the other end in which a bolt with a large enough cap may be threaded, in order to prevent the slices from sliding off at that side.

In addition or as an alternative to having bolts with caps sized as discussed above, a different locking mechanism may be included in a prosthesis assembly. For example, its core, slices and/or bolt(s) may be formed such that threading of at least one of the bolts causes the matching structures of the core and slices to deform and prevent free movement.

As apparent from the above discussions, a surgical kit according to the present disclosure may include at least sone of the following, and optionally a plurality of each: an expandable prosthesis mold configured to be arthroscopically introduced into a joint; an arthroscopic instrument configured to form an ellipsoidal cavity between two interfacing bones of the joint, for receiving the mold; a flowable, curable substance configured for forming the prosthesis inside the mold; an expandable spacer configured when expanded, to maintain the ellipsoidal cavity between the two interfacing bones; an impactor; a pumping system; an arthroscopic extraction instrument; a starter drill configured to drill an initial hole in the joint; a guide cannula configured to be secured relative to the joint and to guide said starter drill into the joint at a predetermined angle and to position the guide wire; a guide wire; a file or a cutter; and a UV curer. If the prosthesis assembly is used, its core, slices and optionally one or more other required parts may be included in the kit. Optionally, one or more suitable screwdrivers and/or hex (Allen) keys may be provided as well.

Various versions and alternatives for element of the kit have been discussed above. Further one or more elements may be included in the kit, as apparent from the above discussions.

In the description and claims of the application, each of the words “comprise” “include” and “have”, and forms thereof, are not necessarily limited to members in a list with which the words may be associated. In addition, where there are inconsistencies between this application and any document incorporated by reference, it is hereby intended that the present application controls.

What is claimed is:

1-70. (canceled)

71. A surgical kit for arthroscopic, in-situ formation of a joint replacement prosthesis, the surgical kit comprising:
an expandable prosthesis mold configured to be arthroscopically introduced into a joint;
at least one arthroscopic instrument configured to form an ellipsoidal cavity between two interfacing bones of the joint, for receiving said mold; and
a first flowable, curable substance configured for forming the prosthesis inside said mold.

72. The surgical kit according to claim 71, wherein said mold is characterized by a smooth, ellipsoidal inner surface, such that an outer surface of said prosthesis, when formed, is smooth and ellipsoidal.

73. The surgical kit according to claim 72, wherein said ellipsoidal inner surface comprises a spheroidal inner surface.

74. The surgical kit according to claim 73, wherein said spheroidal inner surface comprises a spherical inner surface.

75. The surgical kit according to claim 71, wherein said mold is characterized by a final inflated size.

76. The surgical kit according to claim 75, wherein said mold comprises a balloon made of a rigid material.

77. The surgical kit according to claim 75, wherein said mold is made of a non-elastic material.

78. The surgical kit according to claim 71, further comprising an expandable spacer configured to be arthroscopically introduced into the joint, wherein said spacer is configured, when expanded, to maintain the ellipsoidal cavity between the two interfacing bones at least during the formation of the prosthesis.

79. The surgical kit according to claim 78, wherein said mold is provided within said spacer, such that said mold and said spacer are configured to be arthroscopically introduced into the joint together.

80. The surgical kit according to claim 78, wherein said first flowable, curable substance is further configured for forming a prosthetic layer over the prosthesis, inside said spacer.

81. The surgical kit according to claim 78, further comprising a pumping system configured to control inflation of said mold, control injection of said substance into said mold and/or control inflation of said spacer.

82. The surgical kit according to claim 71, further comprising an arthroscopic extraction instrument configured to extract said mold after the prosthesis is formed.

83. The surgical kit according to claim 71, further comprising a guide wire configured to guide surgical tools into said joint.

84. The surgical kit according to claim 83, further comprising starter drill configured to drill an initial hole in the joint and wherein said starter drill comprises an adjustable stopper configured to allow drilling up to a preset depth and/or a convex end surface.

85. The surgical kit according to claim 83, further comprising a convex, expandable reamer configured to form the ellipsoidal cavity between the two bones, wherein said reamer is configured, when forming the ellipsoidal cavity, to ream at least one of the two bones.

86. The surgical kit according to claim 83, further comprising a guide cannula configured to be secured relative to the joint to guide said guide wire into the joint at a predetermined angle and/or configured to guide said reamer into the joint at the predetermined angle over said guide wire.

87. The surgical kit according to claim 71, wherein the expandable prosthesis mold comprises at least one wire, wherein at least a part of said wire is attached to a surface of said mold or embedded in said mold, said wire having a proximal end and a distal end; and wherein at least part of said mold is configured to rip along the path of said wire upon pulling the proximal end of said wire.

88. A minimally-invasive method for in-situ formation of a joint replacement prosthesis, the method comprising:
arthroscopically forming an ellipsoidal cavity between two interfacing bones of the joint; and
arthroscopically forming an ellipsoidal joint replacement prosthesis in the cavity, wherein the forming of said ellipsoidal joint replacement prosthesis comprises arthroscopically introducing an expandable prosthesis mold into said cavity; and injecting a first flowable, curable substance into said mold, thereby forming said ellipsoidal joint replacement prosthesis inside said mold.

89. The method according to claim 88 further comprising, prior to injecting said substance:
arthroscopically introducing an expandable spacer into the joint; and
expanding said spacer to maintain the ellipsoidal cavity between the two interfacing bones at least during the formation of the prosthesis.

90. A joint replacement prosthesis assembly for in-situ formation of a joint replacement prosthesis, the assembly comprising a plurality of parts, each being sized so as to enable its minimally-invasive introduction into a damaged joint, wherein said plurality of parts are configured to be assembled into the joint replacement prosthesis.
